(19) United States
(12) Patent Application Publication
(10) Pub. No.: US 2025/0340592 A1
(43) Pub. Date: Nov. 6, 2025

(54) MAMMALIAN MILK-DERIVED PEPTIDES WITH ANTIMICROBIAL ACTIVITY

(71) Applicant: MATRUBIALS INC., Davis, CA (US)

(72) Inventor: Ishita M. SHAH, Davis, CA (US)

(21) Appl. No.: 18/866,061

(22) PCT Filed: May 18, 2023
(86) PCT No.: PCT/US2023/022790

§ 371 Date: Nov. 14, 2024

Related U.S. Application Data
(60) Provisional application No. 63/343,536, filed on May 18, 2022.

(51) Int. Cl. C07K 7/06; A01N 63/50; A01P 1/00; A61K 8/64; A61K 38/00; A61K 47/10; A61K 47/38; A61P 31/04; A61Q 19/00

(52) U.S. Cl. CPC C07K 7/06; A01N 63/50; A01P 1/00; A61K 8/64; A61K 47/10; A61K 47/38; A61P 31/04; A61Q 19/00; A61K 38/00

(57) ABSTRACT

Provided herein are milk-derived peptide compounds and compositions having antimicrobial activity. The compounds, compositions, and methods are useful as alternatives to existing antibiotic and antimicrobial agents.

### CROSS-REFERENCE TO RELATED APPLICATION(S)

This application is a national phase filing under 35 U.S.C. § 371 of International Application No. PCT/US2023/022790, filed on May 18, 2023, which claims the benefit of U.S. Provisional Application No. 63/343,536 filed on May 18, 2022, the entire contents of which are incorporated by reference.

### STATEMENT REGARDING FEDERALLY SPONSORED RESEARCH OR DEVELOPMENT

This invention was made with government support under 1 R43 AI165105-01A1 awarded by the National Institute of Allergy and Infectious Diseases (NIAID). The government has certain rights in the invention.

### INCORPORATION BY REFERENCE

This application contains a sequence listing entitled “126280_00003_SequenceListing.xml,” being submitted herein in xml format, which was created on Jul. 29, 2023, and is 377,874 bytes in size.

### FIELD

Provided herein are milk-derived peptide compounds and compositions having anti-microbial activity. The compounds, compositions, and methods are useful as alternatives to existing antibiotic and antimicrobial agents.

### BACKGROUND OF THE INVENTION

There is a well-known need to replace the existing antibiotics and the problem continues to worsen. Mammalian milk is a source of antimicrobial compounds. It has been reported that milk of the mammalian mother has been selected through evolution for its ability to protect the infant from bacterial pathogens. Petherick, 2010, Nature, 468, S5-S7; Smilowitz et al., 2014, Annual Review of Nutrition, 34, 143-169; and Walker, 2010, Journal of Pediatrics, 156, S3-S7. Notably, the fragments of milk proteins released during infant digestion have antimicrobial properties. These peptides are typically inactive within the sequence of the parent protein and become active when released by proteolysis. Dallas et al., 2013, Journal of Proteome Research, 12, 2295-2304.

With the increase in resistance to existing antibiotic compositions, milk-derived peptides are a valuable starting point for formulating antimicrobial peptide compositions to address the increasingly critical need for effective antimicrobial compounds.

### SUMMARY OF THE INVENTION

Provided herein are milk-derived peptides including antimicrobial compositions comprising milk-derived peptides having anti-microbial activity. The contemplated invention provides peptides, peptide compositions, peptide variants, and formulations thereof, wherein the peptide originates, or is derived from, milk proteins of various mammals. The disclosed peptides have antimicrobial activity and are useful for therapeutic purposes including the treatment and prevention of primary or secondary diseases caused by bacteria, fungi, viruses, and/or other microbial organisms. The peptides, compositions, and methods are useful as alternatives to existing antibiotic and antimicrobial agents. Advantageously the peptides, compositions, and methods as disclosed herein provide an effective antibiotic peptide treatment of antibiotic resistant strains. Additionally, the disclosed antimicrobial peptides are useful to preserve and stabilize cosmetic formulations.

In one embodiment, a peptide having an amino acid sequence selected from any one of SEQ ID NOS: 1-295. Each peptide may have an amino acid sequence consisting of one of SEQ ID NOS: 1-295.

Provided is a composition comprising a peptide having an amino acid sequence selected from any one of SEQ ID NOS: 1-295. In another embodiment, provided is a (antimicrobial) composition comprising one or more peptides or polypeptides having an amino acid sequence selected from any one of SEQ ID NOS: 1-295.

In other embodiments, a composition comprises a peptide having an amino acid sequence selected from any one of SEQ ID NOS: 1-295 formulated with other excipients. Typically, the composition has antimicrobial activity. In typical embodiments, the antimicrobial composition comprises a peptide having an amino acid sequence selected from SEQ ID NOS: 1-217, 219-254, and 256-295. In exemplary embodiments, the antimicrobial composition comprises a peptide having an amino acid sequence selected from any one of SEQ ID NOS: 1, 6, 11, 16, 21, 26, 31, 36, 41, 76, 106, 141, 142, 177, 178, 213, 218, 219, 220, 255, 256, and 291. In exemplary embodiments, the antimicrobial composition comprises a peptide having an amino acid sequence selected from any one of SEQ ID NOS: 1, 6, 11, 16, 21, 26, 31, 36, 41, 76, 106, 141, 142, 177, 178, 213, 219, 220, 256, and 291. In still other embodiments, the antimicrobial composition comprises a peptide having an amino acid sequence selected from any one of SEQ ID NOS: 1, 6, 11, 16, 21, 26, 31, 36, 41, 76, 106, 141, 142, 177, 178, 213, 218, 219, 220, 255, 256, and 291. In specific embodiments, the antimicrobial composition comprises a peptide having an amino acid sequence selected from any one of SEQ ID NOS: 26, 177, 213, and 291 formulated with excipients as disclosed herein. Examples of excipients include a solvent and a viscosity increasing agent.

In other embodiments, provided is an antimicrobial composition comprising one or more peptides where the one or more peptides have an amino acid sequence selected from any one of SEQ ID NOS: 1-217, 219-254, and 256-295. In other embodiments provided is an antimicrobial composition comprising one or more peptides where the one or more peptides have an amino acid sequence selected from any one of SEQ ID NOS: 1, 6, 11, 16, 21, 26, 31, 36, 41, 76, 106, 141, 142, 177, 178, 213, 218, 219, 220, 255, 256, and 291. In other embodiments, provided is an antimicrobial composition comprising one or more peptides where the one or more peptides have an amino acid sequence selected from any one of SEQ ID NOS: 1, 6, 11, 16, 21, 26, 31, 36, 41, 76, 106, 141, 142, 177, 178, 213, 219, 220, 256, and 291. In still other embodiments, provided is an antimicrobial composition comprising at least two peptides each having an amino acid sequence selected from any one of SEQ ID NOS: 1, 6, 11, 16, 21, 26, 31, 36, 41, 76, 106, 141, 142, 177, 178, 213, 218, 219, 220, 255, 256, and 291.

In additional embodiments, the antimicrobial composition made of a peptide having an amino acid sequence selected from any one of SEQ ID NOS: 1-295 comprises N or C-terminal modifications, PEGylation, glycolsylation, and/or other suitable synthetic modifications. In additional embodiments, the antimicrobial composition comprising one or more peptides, each having an amino acid sequence selected from any one of SEQ ID NOS: 1-295, includes N or C-terminal modifications, PEGylation, glycolsylation, and/or other suitable synthetic modifications.

In additional or alternative embodiments to those disclosed above, the antimicrobial composition made of a peptide having an amino acid sequence selected from any one SEQ ID NOS: 1-295 comprises a viscosity increasing agent. In typical embodiments, a viscosity increasing agent is one or more selected from hydroxypropyl cellulose, hydroxypropyl methylcellulose, carboxymethyl cellulose (CMC), polyvinylpyrrolidone, dextran, and hyaluronic acid. In exemplary embodiments, the viscosity increasing agent is provided at concentrations ranging from 0.4% to 6% based on the total weight or volume of the composition.

In additional or alternative embodiments to those disclosed above, the antimicrobial composition made of one or more peptides, each having an amino acid sequence selected from any one SEQ ID NOS: 1-295, includes a viscosity increasing agent. In typical embodiments, a viscosity increasing agent is one or more selected from hydroxypropyl cellulose, hydroxypropyl methylcellulose, polyvinylpyrrolidone, dextran, and hyaluronic acid. In exemplary embodiments, the viscosity increasing agent is provided at concentrations ranging from 0.4% to 6% based on the total weight or volume of the composition.

In additional or alternative embodiments, the antimicrobial composition comprises a solvent. In exemplary embodiments, a solvent is selected from one or more of glycerin, ethanol, methanol, propylene glycol, and/or isopropanol. In exemplary embodiments, the antimicrobial composition comprises a solvent selected from one or more of glycerin, propylene glycol, DMSO, and isopropanol in a concentration ranging from 0.1 to 30%; or the antimicrobial composition comprises a solvent selected from methanol and ethanol at a concentration up to 99%.

In exemplary embodiments, a composition having antimicrobial activity comprises a peptide having an amino acid sequence selected from any of SEQ ID NOS: 1-295 formulated with a solvent and a viscosity increasing agent. For example, the composition having antimicrobial activity comprises a peptide having an amino acid sequence selected from any of SEQ ID NOS: 1-295 formulated with a solvent and a viscosity increasing agent. For example, the solvent may be selected from glycerin, propylene glycol, and isopropanol in a concentration ranging from 0.1 to 30% or selected from methanol and ethanol at a concentration up to 99% and the viscosity increasing agent may be selected from hydroxypropyl cellulose, hydroxypropyl methylcellulose, carboxymethyl cellulose (CMC), polyvinylpyrrolidone, dextran, or hyaluronic acid. For example, the concentration of the viscosity increasing agent is from about 0.4% to 6% based on the total weight or volume of the composition. In further examples, the composition having antimicrobial activity comprises a peptide having an amino acid sequence selected from any one of SEQ ID NOS: 1-295, formulated with a solvent and carboxymethyl cellulose. More specifically, a composition having antimicrobial activity comprises a peptide having an amino acid sequence selected from one of SEQ ID NOS: 26, 177, 213, and 291, corresponding to peptides M6, M14, M16, and M22, respectively, as disclosed herein.

In additional or alternative embodiments to those disclosed above, the antimicrobial composition made of a peptide having an amino acid sequence selected from any one SEQ ID NOS: 1-295 comprises one or more organic acids selected from citric acid, malic acid, tartaric acid, acetic acid, butyric acid, phosphoric acid, lactic acid, indole lactic acid, and/or indole ethanol. In some additional embodiments, the antimicrobial composition comprises additional salt in combination with the one or more organic acids for specific pH formulations.

In additional or alternative embodiments to those disclosed above, the antimicrobial composition made of one or more peptides, each having an amino acid sequence selected from any one SEQ ID NOS: 1-295, comprises one or more organic acids selected from citric acid, malic acid, tartaric acid, acetic acid, butyric acid, phosphoric acid, indole lactic acid, and/or indole ethanol. In some additional embodiments, the antimicrobial composition comprises additional salt in combination with the one or more organic acids for specific pH formulations.

In additional or alternative embodiments to those disclosed above, the antimicrobial composition made of a peptide having an amino acid sequence selected from any one SEQ ID NOS: 1-295 comprises a humectant. In additional or alternative embodiments to those disclosed above, the humectant is one or more selected from sorbitol, glycerol, propylene glycol, sorbitol, seaweed, urea, peptides, amino acids, and honey. In additional or alternative embodiments to those disclosed above, the humectant is sorbitol or glycerol. In additional or alternative embodiments to those disclosed above, the antimicrobial composition comprises a humectant together with a preservative selected from benzoic acid, benzyl alcohol, propylparaben, methyl paraben, and/or phenoxyethanol.

In additional or alternative embodiments to those disclosed above, the antimicrobial composition made of one or more peptides having an amino acid sequence selected from any one SEQ ID NOS: 1-295 comprises a humectant. In exemplary embodiments, the humectant is sorbitol or glycerol. In exemplary embodiments, the antimicrobial composition comprises a humectant together with a preservative selected from benzoic acid, benzyl alcohol, propylparaben, methyl paraben, and/or phenoxyethanol.

In additional or alternative embodiments to those disclosed above, the antimicrobial composition made of one or more peptides having an amino acid sequence selected from any one of SEQ ID NOS: 1-295 is formulated as a spray, gel, ointment, wash, cream, or insert tablet.

In additional or alternative embodiments to those disclosed above, the antimicrobial composition made of a peptide having an amino acid sequence selected from any one of SEQ ID NOS: 1-295 also comprises an acid where the acid can be in a salt form, a free acid form, or as any ester form thereof.

In additional or alternative embodiments to those disclosed above, the antimicrobial composition made of one or more peptides having an amino acid sequence selected from any one of SEQ ID NOS: 1-295 also comprises an acid where the acid can be in a salt form, a free acid form, or as any ester form thereof.

In additional or alternative embodiments to those disclosed above, the antimicrobial composition made of one or more peptides having an amino acid sequence selected from any one of SEQ ID NOS: 1-295, also comprises a buffering agent. In exemplary embodiments, the buffering agent is added in a range of 1 to 300 mM. In exemplary embodiments, the buffering agent is one selected from acetate, succinate, lactate, fumarate and any additional monocarboxylates and dicarboxylates.

In some embodiments, a method of treating a microbial infection comprises administering to a subject suffering from a microbial infection, a composition comprising one or more peptides having an amino acid sequence selected from any one of SEQ ID NOS: 1-295. In typical embodiments, a method of treating a microbial infection comprises administering a composition comprising a peptide having an amino acid sequence selected from any one of SEQ ID NOS: 1-140, 142-217, 220-254, and/or 256-295. For example, a method of treating a microbial infection in a subject, comprises administering to the subject in need thereof, a composition comprising a peptide having an amino acid sequence selected from any of SEQ ID NOS: 26, 177, 213, and 291. A microbial infection comprises a bacterial infection, a fungal infection, and/or a viral infection. In some embodiments, the microbial infection is a bacterial infection.

In certain embodiments, the polypeptide sequence is combined with additional polypeptide sequences and one or more solvents, one or more viscosity increasing agents and one or more organic acids or salts for desired formulation.

In certain embodiments, the administration is through a cell harboring the polynucleotide sequence that would encode the polypeptide sequence for delivery for human or non-human application.

In some embodiments, the administration can be topical, intravenous, intraperitoneal, subcutaneous, intramuscular, rectal, nasal, oral for direct association with human or non-human animal or on a surface (e.g., a medical device, or a catheter).

In some embodiments, the peptide is a peptidomimetic of the peptide having an amino acid sequence selected from any one of SEQ ID NOS: 1-295.

### BRIEF DESCRIPTION OF DRAWINGS

FIG. 1. (SEQ ID NO: 296) shows the amino acid sequence of the camel K-casein protein.

FIG. 2. (SEQ ID NO: 297) shows the amino acid sequence of the human Lactotransferrin protein.

FIG. 3. (SEQ ID NO: 298) shows the amino acid sequence of the goat «S2-casein protein.

FIG. 4. (SEQ ID NO: 299) shows the amino acid sequence of the human Lactase-phlorizin hydrolase protein.

FIG. 5. (SEQ ID NO: 300) shows the amino acid sequence of the goat β-casein protein.

FIG. 6. (SEQ ID NO: 301) shows the amino acid sequence of the bovine Lysozyme C isozyme protein.

FIG. 7. depicts the origin, sequence, predicted charge of the first polypeptide of each of the polypeptide variant groups M1-M19, M21, and M22 (SEQ ID NOS: 1, 6, 11, 16, 21, 26, 31, 36, 41, 76, 106, 141, 142, 177, 178, 213, 218, 219, 220, 256, or 291), as disclosed herein.

FIG. 8. shows images from time-kill assays, indicating that the polypeptides (MI (SEQ ID NO: 1); M2 (SEQ ID NO: 6); M3 (SEQ ID NO: 11); M4 (SEQ ID NO: 16); M5 (SEQ ID NO: 21); M6 (SEQ ID NO: 26); M7 (SEQ ID NO: 31); and M8 (SEQ ID NO: 36) target G. vaginalis and L. iners, but not L. crispatus, as indicated.

FIG. 9. depicts the antibiotic activity (in concentration of polypeptide at 0.375 mg/ml (****), 0.75 mg/ml (***), 1.5 mg/ml (**), or 3 mg/ml (*), as indicated) for each of polypeptides (SEQ ID NO: 1 (M1); SEQ ID NO: 6 (M2); SEQ ID NO: 11 (M3); SEQ ID NO: 16 (M4); SEQ ID NO: 21 (M5); SEQ ID NO: 26 (M6); SEQ ID NO: 31 (M7); SEQ ID NO: 36 (M8); SEQ ID NO: 41 (M9); SEQ ID NO: 76 (M10); SEQ ID NO: 106 (M11); SEQ ID NO: 141 (M12); SEQ ID NO: 142 (M13); SEQ ID NO: 177 (M14); SEQ ID NO:178 (M15); SEQ ID NO: 213 (M16); SEQ ID NO: 219 (M18), SEQ ID NO: 220 (M19); SEQ ID NO: 256 (M21); and SEQ ID NO: 291 (M22)) against P. aeruginosa and S. aureus MRSA, as indicated.

FIGS. 10A, 10B, and 10C are graphs depicting bacterial viability in colony forming units (CFU) per milliliter (ml) over time (minutes) in the presence of select polypeptides: SEQ ID NO: 26 (M6); SEQ ID NO: 31 (M7); SEQ ID NO: 177 (M14); SEQ ID NO: 213 (M16); and SEQ ID NO: 291 (M22) in comparison to Indolicidin (IND) in the presence of P. aeruginosa (FIG. 10A), S. aureus MRSA (FIG. 10B), and S. epidermidis (FIG. 10C), respectively.

FIGS. 11A, 11B, and 11C depict the retention of antimicrobial activity of the indicated polypeptides SEQ ID NO: 26 (M6); SEQ ID NO: 31 (M7); SEQ ID NO: 177 (M14); SEQ ID NO: 213 (M16); or SEQ ID NO: 291 (M22), each in a composition with the formulation of 20% propylene glycol and 2% hydroxypropyl cellulose (F is the formulation without a peptide), in the presence of P. aeruginosa (FIG. 11A), S. aureus MRSA (FIG. 11B), or S. epidermidis (FIG. 11C), respectively, as indicated.

FIGS. 12A, 12B, 12C, and 12D depict the retention of antimicrobial activity of the indicated peptides M6 (SEQ ID NO: 26), M14 (SEQ ID NO:177), M16 (SEQ ID NO: 213) and M22 (SEQ ID NO: 291), with each peptide formulated in a composition with 20% propylene glycol and 2% hydroxypropyl cellulose (F is the formulation without a peptide), in the presence of G. vaginalis (FIG. 12A), L. iners (FIG. 12B), L. crispatus (FIG. 12C), or A. baumannii (FIG. 12D), from 0 minutes (min) to 60 minutes, as indicated, where the amount of growth is indicated as: 4 is full growth; 3 is too many colonies to count; 2 is countable colonies; and 1 is no growth.

FIG. 13 depicts the retention of antimicrobial activity of the indicated peptides M6 (SEQ ID NO: 26), M14 (SEQ ID NO:177), M16 (SEQ ID NO: 213) and M22 (SEQ ID NO: 291), with each peptide formulated in a composition with 20% propylene glycol and 2% hydroxypropyl cellulose (F is the formulation without a peptide), in the presence of S. aureus MRSA, from 0 minutes (min) to 60 minutes, as indicated, where the amount of growth is indicated as: 4 is full growth; 3 is too many colonies to count; 2 is countable colonies; and 1 is no growth.

FIG. 14A lists eight of the most peptide-resistant strains from the MRSN panel of 100 P. aeruginosa strains and their sensitive(S) or resistant (R) growth phenotype with respect to the indicated antibiotic.

FIG. 14B depicts the growth of the eight peptide resistant P. aeruginosa strains from FIG. 14B at 0.003% (30 μg/ml final concentration) in the presence of M14 (SEQ ID NO: 17) and or M16 (SEQ ID NO: 213) after 24 hours, where the amount of growth is indicated as: 4 is full growth; 3 is too many colonies to count; 2 is countable colonies; and 1 is no growth.

FIGS. 15A-15B show the growth of P. aeruginosa strain NR-51553 of FIG. 14A in a modified CLSI-based broth dilution assay, in the presence of peptide M16 (SEQ ID NO: 213) in 0.01×MH broth (FIG. 15A) and in the presence of peptide M16 in 0.01×MH broth and 0.5% carboxymethyl cellulose (CMC) (FIG. 15B), where the amount of growth is indicated as: 4 is full growth; 3 is too many colonies to count; 2 is countable colonies; and 1 is no growth.

FIG. 16 depicts the growth of S. aureus MRSA in a modified CLSI-based broth dilution assay, in the absence or presence of each of the indicated peptides: M6 (SEQ ID NO: 26), M14 (SEQ ID NO:177), and M16 (SEQ ID NO: 213), where 250 μg/ml of M14 peptide in 0.01×MH broth at a time of 4 hours is more efficacious compared to 250 μg/ml of M16 at a time of 24 hours, where the amount of growth is indicated as: 4 is full growth; 3 is too many colonies to count; 2 is countable colonies; and 1 is no growth.

FIG. 17 depicts the growth of A. baumanii in a modified CLSI-based broth dilution assay in the absence or presence of each of the indicated peptides: M14 (SEQ ID NO:177) and M16 (SEQ ID NO: 213), where 250 μg/ml of M16 peptide in 0.01×MH broth at a time of 4 hours is more efficacious compared to 250 μg/ml of M14 at a time of 24 hours, where the amount of growth is indicated as: 4 is full growth; 3 is too many colonies to count; 2 is countable colonies; and 1 is no growth.

FIG. 18A shows the ex vivo growth of E. faecalis in HBT bilayer agar obtained from vaginal swabs from patients with symptoms of bacterial vaginosis, the E. faecalis growth is in the absence or presence of peptides M6 (SEQ ID NO: 26), M14 (SEQ ID NO: 177), and the standard of care (SOC) antibiotics metronidazole (MET) and clindamycin (CLIN), as indicated.

FIG. 18B shows the ex vivo growth of C. albicans on MRS agar obtained from a vaginal swab from a patient, the C. albicans growth is in the absence or presence of peptides M6 (SEQ ID NO: 26), M14 (SEQ ID NO:177), and the standard of care (SOC) antibiotics metronidazole (MET) and clindamycin (CLIN), as indicated.

FIG. 19 is a graph of a cytotoxicity assay in vaginal epithelial cells (VK2/E6E7) and cervical cells (HeLa) in the absence (None) or presence of 1 mg/ml, 5 mg/ml, and 10 mg/ml concentrations of peptides M6 (SEQ ID NO: 26) and M14 (SEQ ID NO: 177) with Ethanol as a positive control for cytotoxicity, where the number of viable cells is proportional to the calcein fluorescence units (FU), or cell death is observed as the decrease in calcein FU.

FIG. 20 is a graph of a biofilm disruption assay of G. vaginalis biofilms in the presence of peptide M6 (SEQ ID NO: 26), where a decrease in the amount of crystal violet dye corresponds to disruption of the biofilm, and disruption of G. vaginalis is observed at concentrations of 0.02 mg/ml, 0.2 mg/ml, and 2.0 mg/ml of M6 (SEQ ID NO: 26), as indicated.

FIGS. 21A, 21B, and 21C are graphs showing the percentage (%) of HIV infection in TZM-bl cells exposed to 100, 1000, or 5000 μg/ml of peptide M6 (SEQ ID NO: 26) with or without HIV-1 for 48 hours, where in addition to the M6 peptide, the cells are: i) co-exposed to HIV or media only (FIG. 21A); ii) pre-exposed to the M6 peptide or media for 6 hours; or iii) post-exposed to the M6 peptide after exposed to HIV for 2 hours, and then exposed to HIV and the M6 peptide for 36 hours.

FIG. 22 is graph showing the amount (%) reduction of HSV DNA in HSV-2G infected HEC1A cells infected with HSV-2G virus at the indicated amounts of multiplicity of infection (MOI), followed by virus removal and incubation with peptide M6 (SEQ ID NO: 26) at 5 mg/ml for 3 days, where the amount of HSV DNA is then quantified relative to HSV-2G infected HEC1A cells that were not incubated with peptide M6 (SEQ ID NO: 26).

### DETAILED DESCRIPTION OF INVENTION

The peptide sequences or analogs thereof derived from mammalian milk proteins disclosed herein have activity against antimicrobial pathogens. These peptides are useful anti-infectives for inhibiting or preventing pathogenic growth. These peptides may be administered alone or in formulated compositions (e.g., with excipients). The formulated compositions comprising the contemplated peptides include pharmaceutical compositions and/or cosmetic formulations. Contemplated methods include treating a subject (human or animal) or a surface (e.g., a medical device) with a composition including a peptide as disclosed herein, to inhibit or treat (by cell death) pathogenic microbial growth on (or in) the subject or surface. Contemplated methods also include preserving (e.g., increasing the shelf-life of) a cosmetic composition by adding one or more of the contemplated peptides to the cosmetic composition, thereby adding an antimicrobial (e.g., bactericidal and/or anti-fungal) ingredient to the cosmetic composition.

### Definitions

As used herein, with respect to bacteria disclosed herein, P. aeruginosa refers to Pseudomonas aeruginosa, S. aureus refers to Staphylococcus aureus, S. aureus MRSA refers to methicillin-resistant S. aureus, S. epidermidis refers to Staphylococcus epidermidis, G. vaginalis refers to Gardnerella vaginalis, L. iners refers to Lactobacillus iners, L. crispatus refers to Lactobacillus crispatus, A. baumannii refers to Acinetobacter baumannii, and E. faecalis refers to Enterococcus faecalis.

As used herein, 100 MRSN panel P. aeruginosa refers to a reference panel of 100 strains of P. aeruginosa collected by the Multi-drug resistant organism Repository and Surveillance Network (MRSN).

As used herein, with respect to yeast disclosed herein, Candida albicans may be referred to as C. albicans.

As used herein, with respect to viruses disclosed herein, HIV refers to human immunodeficiency virus and HSV refers to herpes simplex virus.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

The terms “peptidomimetic” and “mimetic” refer to a synthetic chemical compound that has substantially the same functional characteristics of a naturally or non-naturally occurring polypeptide, but different (though typically similar) structural characteristics. Peptide analogs are commonly used in the field as non-peptide active compounds (e.g., drugs) with properties analogous to those of a template peptide. Such non-peptide compounds are termed “peptide mimetics” or “peptidomimetics” (Fauchere, J. Adv. Drug Res. 15:29 (1986); Veber and Freidinger, TINS p. 392 (1985); and Evans et al. J. Med. Chem. 30:1229 (1987)). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., SEQ ID NO:1) such as found in a polypeptide of interest, but have one or more peptide linkages optionally replaced by a linkage selected from the group consisting of, e.g., —CH2NH—, —CH2S-, —CH2-CH2-, —CH═CH— (cis and trans), —COCH2-, —CH(OH)CH2-, and —CH2SO—. A mimetic can be either entirely composed of synthetic, non-natural analogues of amino acids, or, is a chimeric molecule of partly natural peptide amino acids and partly non-natural analogs of amino acids. A mimetic can also incorporate any amount of natural amino acid conservative substitutions as long as such substitutions also do not substantially alter the mimetic's structure and/or (e.g., antibacterial) activity.

The term “amino acid” refers to naturally occurring and synthetic amino acids, as well as amino acid analogs that function in a manner similar to naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) as well as pyrrolysine, pyrroline-carboxy-lysine, and selenocysteine.

As used herein, the term “substantial identity” or “substantially identical,” used in the context of nucleic acids or polypeptides, refers to a sequence that has at least 50% sequence identity with a reference sequence. Alternatively, percent identity is any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as described using standard parameters for a BLAST. Accordingly, embodiments of the present disclosure provide for antimicrobial (e.g., antibacterial) peptides that are substantially identical to any of SEQ ID NOS: 1-295 if it has at least 50%, 55%, 60%, 65%, 70%, 75%, 80%, 85%, 90%, 91%, 92%, 93%, 94%, 95%, 96%, 97%, 98% or 99% sequence identity.

As used herein, a polynucleotide or polypeptide sequence is “heterologous” to a cell if it originates from a different cell, or, if from the same cell, is modified from its original form. For example, when a first amino acid sequence in a protein is said to be heterologous to a second amino acid sequence in the same protein, it means that the first amino acid is from a first cell or is non-naturally-occurring whereas the second amino acid is from a second cell or is modified from its original form.

As used herein, “pharmaceutically acceptable salt” includes salts with an inorganic or organic acid. In some embodiments, salts with an inorganic acid include, without limitation, hydrochloride, hydrobromide, nitrate, sulfate, phosphate, and the like. In some embodiments, salts with an organic acid include, without limitation, citric acid (citrate), malic acid (maleate), tartaric acid (tartrate), acetic acid (acetate), butyric acid (butyrate), phosphoric acid (phosphate), indole lactic acid (indole lactate), lactic acid (lactate), formic acid (formate), trifluoroacetic acid (trifluoroacetate), propionic acid (propionate), oxalic acid (oxalate), ascorbic acid (ascorbate), fumaric acid (fumarate), malonic acid (malonate), methanesulfonic acid (methanesulfonate), benzenesulfonic acid (benzenesulfonate), p-toluenesulfonic acid (p-toluenesulfonate), and the like.

As used herein, “a cosmetically acceptable salt” includes salts with an inorganic or organic acid. In some embodiments, salts with an inorganic acid include, without limitation, hydrochloride, hydrobromide, nitrate, sulfate, phosphate, and the like. In some embodiments, salts with an organic acid include, without limitation, citric acid (citrate), malic acid (maleate), tartaric acid (tartrate), acetic acid (acetate), butyric acid (butyrate), phosphoric acid (phosphate), indole lactic acid (indole lactate), lactic acid (lactate), formic acid (formate), trifluoroacetic acid (trifluoroacetate), propionic acid (propionate), oxalic acid (oxalate), ascorbic acid (ascorbate), fumaric acid (fumarate), malonic acid (malonate), methanesulfonic acid (methanesulfonate), benzenesulfonic acid (benzenesulfonate), p-toluenesulfonic acid (p-toluenesulfonate), and the like.

As used in this specification and the appended claims, reference to “about” a value or parameter herein includes (and describes) variations that are directed to that value or parameter per se. For example, description referring to “about X” includes description of “X”.

As used in this specification and the appended claims, and unless otherwise specified, the terms “about” and “approximately,” when used in connection with various terms such as temperatures, doses, amounts, or weight percent of ingredients of a composition or a dosage form, mean e.g. a temperature, dose, amount, or weight percent that is recognized by those of ordinary skill in the art to provide an effect equivalent to that obtained from the specified temperature dose, amount, or weight percent. Specifically, the terms “about” and “approximately,” when used in this context, contemplate a temperature, dose, amount, or weight percent, etc. within 15%, within 10%, within 5%, within 4%, within 3%, within 2%, within 1%, or within 0.5% of the specified temperature, dose, amount, or weight percent, etc.

As used in this specification and the appended claims, it is understood that a concentration of 1.0% refers to weight per volume, such that 1.0% 10 mg/ml, and a concentration of 0.5% is 5 mg/ml, and so on.

As used in this specification and the appended claims, the singular forms “a,” “an,” and “the” include plural referents unless the context clearly dictates otherwise. For example, “a peptide” can mean “one or more peptides,” which are independently selected; “a solvent” can mean “one or more solvents;” “an humectant” can mean “one or more humectants;” and the like.

### DETAILED DESCRIPTION

The contemplated peptides as disclosed herein correspond to one of 22 peptide groups (i.e., 22 peptide families), referred to herein as MI to M22. For example, the peptides represented by SEQ ID NOs: 1-295 encompass peptides from each of M1 to M22 as set forth in Tables 1A-1L.

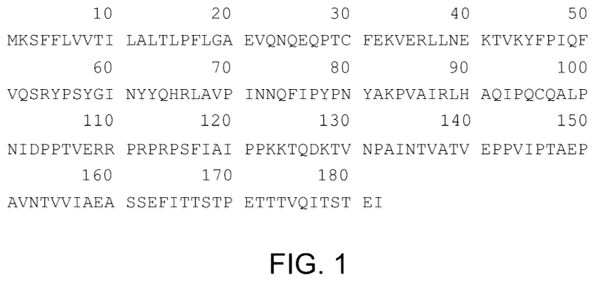

In some embodiments, peptides are derived from naturally occurring milk proteins. As such, the “derived” peptides are mutants, variants, or unique fragments of naturally occurring proteins, and as such, are not known to exist in nature as presently disclosed. The amino acid sequences of the milk proteins from which each of the contemplated peptides is derived are set forth in FIGS. 1-6. The sequences for the camel K-casein protein, the human Lactotransferrin protein, the goat «S2-casein protein, the human Lactase-phlorizin hydrolase, the goat β-casein protein, and the bovine Lysozyme C isozyme protein are shown, respectively.

Contemplated compositions may include combinations of one or more peptides as disclosed herein. For example, a composition may include at least two peptides having an amino acid sequence selected from any one of SEQ ID NOS: 1-295. More specifically, contemplated compositions include at least two peptides having an amino acid sequence selected from any one of SEQ ID NOS: 1, 6, 11, 16, 21, 26, 31, 36, 41, 76, 106, 142, 177, 178, 213, 218, 219, 220, 255, 256, and 291.

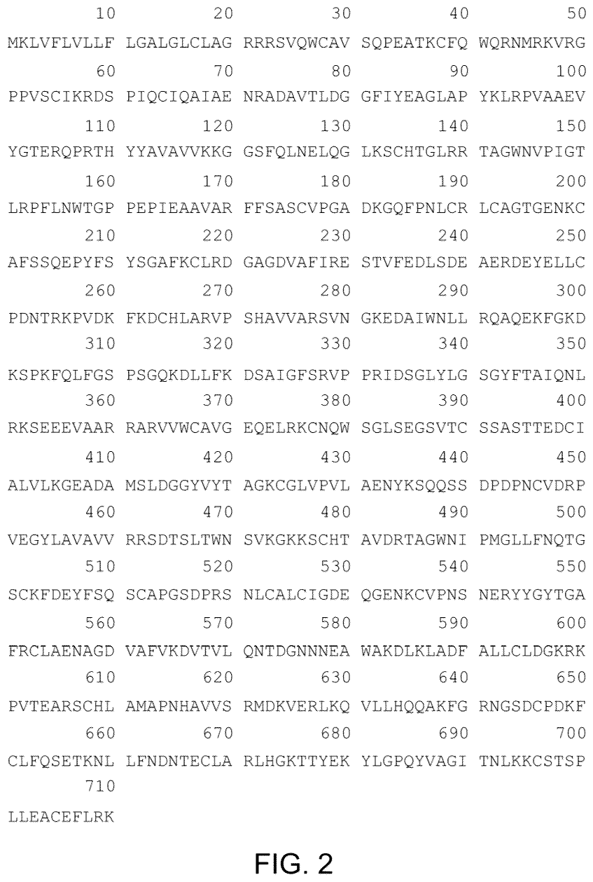

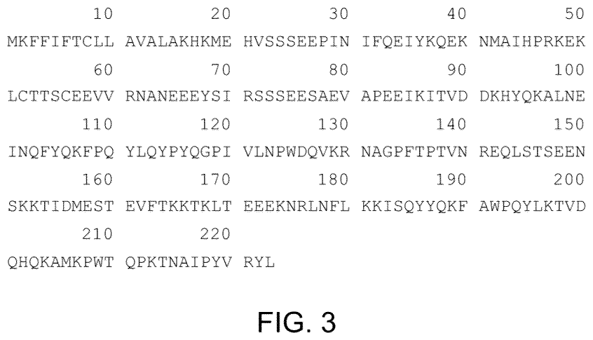

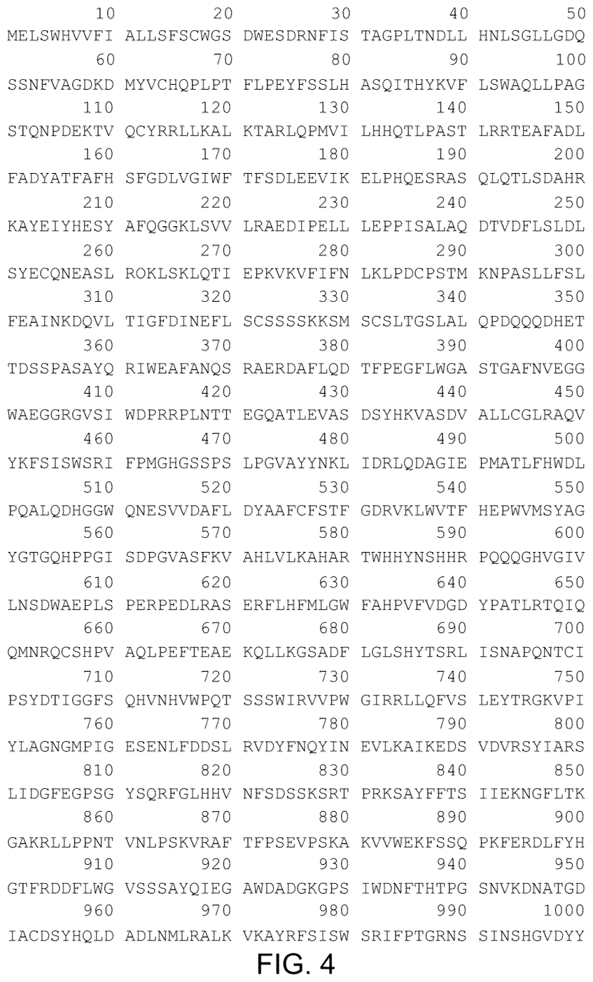

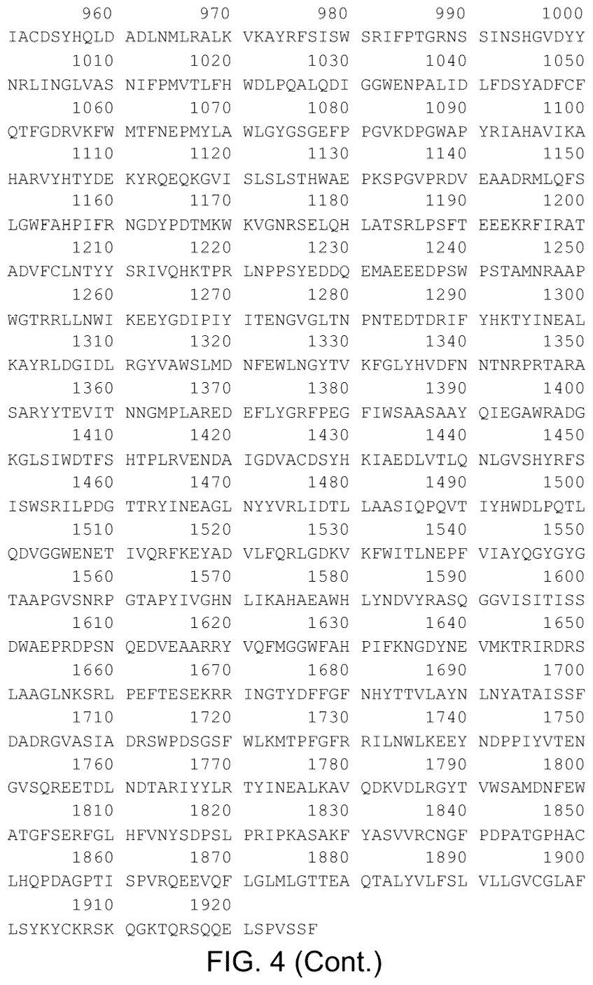

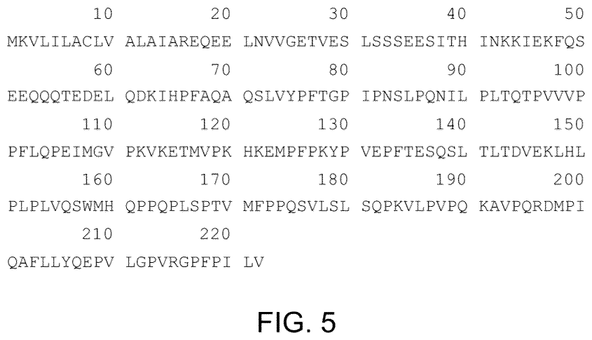

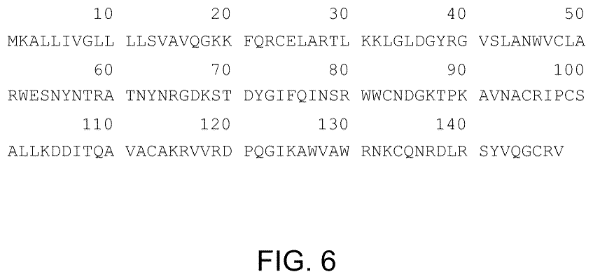

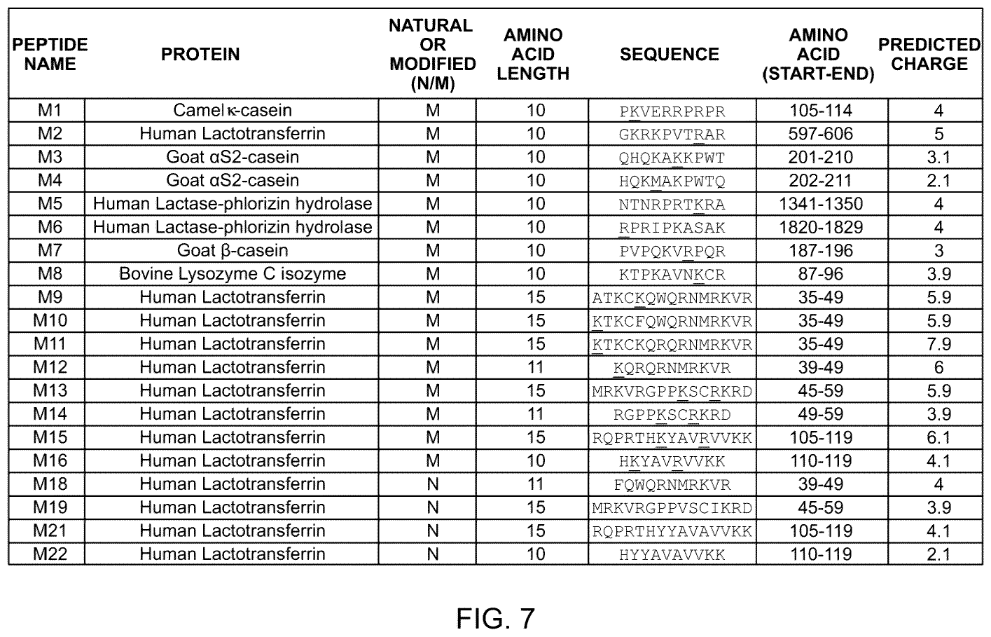

With reference to FIG. 7, the native protein for each peptide group MI to M22 is provided (“PROTEIN”) and the amino acid sequence of the first peptide for each group is provided (“SEQUENCE”). If the first peptide sequence is modified from the native protein as indicated (“M”), the amino acid(s) that is/are modified is underlined. As shown, the M1 peptide group corresponds to camel K-casein amino acid sequence, UniProtKB-P79139 (CASK_CAMDR) (FIG. 1). The M2 and M9-M22 peptide groups correspond to the human Lactotransferrin amino acid sequence, UniProtKB-P02788 (TRFL_HUMAN) (FIG. 2). The M3 and M4 peptide groups correspond to the goat «S2-casein amino acid sequence, UniProtKB-P33049 (CASA2_CAPHI) (FIG. 3). The M5 and M6 peptide groups correspond to the human Lactase-phlorizin hydrolase amino acid sequence, UniProtKB-P09848 (LPH_HUMAN) (FIG. 4). The M7 peptide group corresponds to goat β-casein amino acid sequence, UniProtKB-P33048 (CASB_CAPHI) (FIG. 5). The M8 peptide group corresponds to bovine Lysozyme C isozyme amino acid sequence, UniProtKB-Q6B411 (LYSM_BOVIN) (FIG. 6).

With reference to FIG. 7 together with the peptide sequences set forth in Tables 1A-1L, the specific distinctions of the contemplated peptides represented by SEQ ID NO: 1-295 are disclosed. For example, the contemplated peptides may comprise amino acid sequences having 0, 1, 2, 3 or 4 amino acid changes compared to a native peptide sequence.

The contemplated milk-derived peptides may be at least 8 amino acids in length, and may be up to 200 amino acids in length. In exemplary embodiments, the peptides are from 8 to 200, 8 to 100, 8 to 50, 8 to 40, 8 to 30, 8 to 20, 8 to 15, 8 to 14, 8 to 13, 8 to 12, 8 to 11, 8 to 10, 8 to 9, or 8 amino acids in length. In exemplary embodiments, the peptides are from 8 to 15 amino acids in length.

Additionally, the milk-derived protein made be fused to an independent (non-milk derived) heterologous protein or peptide (e.g., a labeling or purification tag). For example, with respect to SEQ ID NOs. 1-295, each of these peptides ranging in length from 8 to 15 amino acids may be fused to a heterologous peptide or protein.

In some embodiments, the contemplated peptides have antimicrobial activity. The antimicrobial activity comprises any decrease in microbial growth. In exemplary embodiments, an antimicrobial composition comprises a peptide having an amino acid sequence selected from any one of SEQ ID NOS: 1-295. In more typical embodiments, an antimicrobial composition comprises a peptide having an amino acid sequence selected from any one of SEQ ID NOS: 1-217, 220-254, and/or 256-295. In exemplary embodiments, an antimicrobial composition comprises a peptide having an amino acid sequence selected from any one of SEQ ID NOS: 1, 6, 11, 16, 21, 26, 31, 36, 41, 76, 106, 141, 142, 177, 178, 213, 218, 219, 220, 255, 256, and 291. In exemplary embodiments, an antimicrobial composition comprises a peptide having an amino acid sequence selected from any one of SEQ ID NOS: 1, 6, 11, 16, 21, 26, 31, 36, 41, 76, 106, 142, 177, 178, 213, 220, 256, and 291. In further examples, an antimicrobial composition comprises a peptide having an amino acid sequence selected from SEQ ID NOS: 26, 177, 213, and 291.

Notably, antimicrobial peptide compositions for inhibiting microbial growth on a surface or on skin (e.g., in a cosmetic composition or wound composition), the peptide concentration may be as high as 50 mg/ml. Accordingly, the antimicrobial composition comprises a peptide having an amino acid sequence selected from any one of SEQ ID NOS: 1-295, wherein the amount of peptide in the antimicrobial compensation ranges from about 0.020 mg/ml up to 50 mg/ml of peptide. In exemplary embodiments, the antimicrobial composition for inhibiting or treating microbial growth on a surface or on skin comprises a peptide having an amino acid sequence selected from any one of SEQ ID NOS: 1-295, wherein the amount of peptide in the antimicrobial compensation ranges from about 0.1 mg/ml up to 45 mg/ml; about 0.5 mg/ml to about 40 mg/ml; 1 mg/ml to about 50 mg/ml; 1 mg/ml to about 40 mg/ml; about 1 mg/ml to about 30 mg/ml; about 1 mg/ml to about 20 mg/ml; about 5 mg/ml to about 50 mg/ml; about 5 mg/ml to about 25 mg/ml; about 5 mg/ml to about 20 mg/ml; about 5 mg/ml to about 15 mg/ml; about 5 mg/ml to about 10 mg/ml; or about 10 mg/ml to about 40 mg/ml.

For administration of an antimicrobial peptide composition to a subject, the peptide concentration may be from about 0.02 mg/ml (0.002%) up to 20 mg/ml (2.0%). In exemplary embodiments, the antimicrobial composition for inhibiting or treating microbial growth in a subject, comprises a peptide having an amino acid sequence selected from any one of SEQ ID NOS: 1-295, wherein the amount of peptide in the antimicrobial compensation ranges from about 0.1 mg/ml up to 20 mg/ml; about 0.1 mg/ml to about 10 mg/ml; 0.1 mg/ml to about 8 mg/ml; 0.1 mg/ml to about 7 mg/ml; about 0.1 mg/ml to about 6 mg/ml; about 0.1 mg/ml to about 5 mg/ml; about 0.1 mg/ml to about 4 mg/ml; about 0.1 mg/ml to about 3.5 mg/ml; about 0.1 mg/ml to about 3.0 mg/ml; about 0.1 mg/ml to about 2.5 mg/ml; about 0.1 mg/ml to about 2.0 mg/ml; or about 0.10 mg/ml to about 1.0 mg/ml. In other exemplary embodiments, the antimicrobial composition for inhibiting or treating microbial growth in a subject, comprises a peptide having an amino acid sequence selected from any one of SEQ ID NOS: 1-295, wherein the amount of peptide in the antimicrobial compensation ranges from about 0.02 mg/ml up to about 5 mg/ml; about 0.02 mg/ml to about 4 mg/ml; about 0.02 mg/ml to about 3 mg/ml; about 0.02 to about 2 mg/ml; about 0.50 to about 10 mg/ml; about 0.5 mg/ml to about 8 mg/ml; about 0.5 to about 6 mg/ml; about 0.5 mg/ml to about 4 mg/ml; or about 0.5 mg/ml to about 2 mg/ml.

The antimicrobial composition comprising a peptide having an amino acid sequence selected from of SEQ ID NOS: 26, 177, 213, and/or 291 is formulated with a viscosity increasing agent, as disclosed herein.

In some embodiments, the contemplated peptides have antimicrobial activity. The antimicrobial activity includes any decrease in microbial growth. In exemplary embodiments, an antimicrobial composition comprises one or more peptides or polypeptides each having an amino acid sequence selected from any one of SEQ ID NOS: 1-295. In more typical embodiments, an antimicrobial composition comprises one or more peptides each having an amino acid sequence selected from any one of SEQ ID NOS: 1-217, 220-254, and/or 256-295. In exemplary embodiments, an antimicrobial composition comprises one or more peptides each having an amino acid sequence selected from any one of SEQ ID NOS: 1, 6, 11, 16, 21, 26, 31, 36, 41, 76, 106, 141, 142, 177, 178, 213, 218, 219, 220, 255, 256, and 291. In exemplary embodiments, an antimicrobial composition comprises one or more peptides each having an amino acid sequence selected from any one of SEQ ID NOS: 1, 6, 11, 16, 21, 26, 31, 36, 41, 76, 106, 142, 177, 178, 213, 220, 256, and 291.

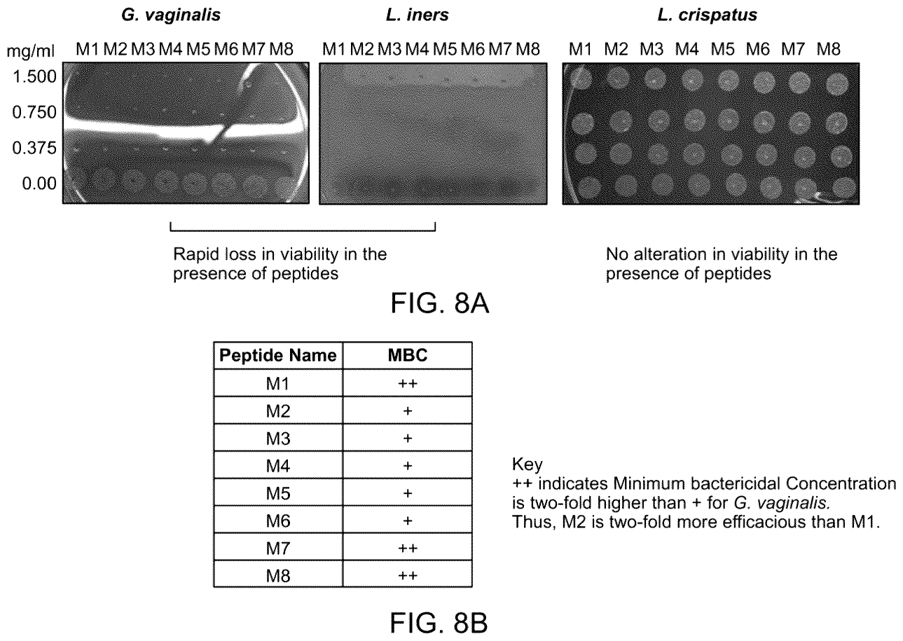

In some embodiments, the contemplated peptides have advantageously selective antimicrobial activity. Notably, as shown, in FIG. 8, exemplary peptides (SEQ ID NOS: 1, 6, 11, 16, 21, 26, 31, and 36) are capable of killing major pathogens including P. aeruginosa, S. aureus, including MRSA, S. epidermidis, G. vaginalis, and advantageously, not L. crispatus, a common strain found in a healthy vagina.

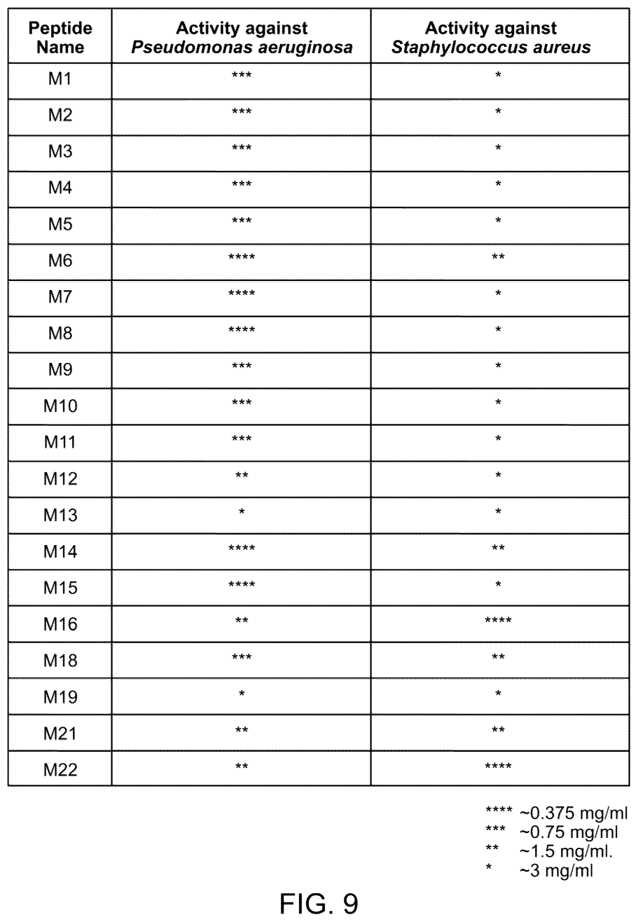

In some embodiments, the contemplated peptides have antimicrobial activity against one or more than one pathogen, and/or the contemplated peptides have increased potency against one or more than one pathogen. For example, as shown in FIG. 9, exemplary peptides M6, M7, M8, M14, and M15 (SEQ ID NOS: 26, 31, 36, 177 and 178, respectively) are more effective in reducing the viability of P. aeruginosa as compared to peptides M1, M2, M3, M4, and M5 (SEQ ID NOS: 1, 6, 11, 16, and 21, respectively).

Contemplated compositions comprise combinations of at least two of the peptides as disclosed herein. For example, an antimicrobial composition comprises at least two peptides, each having an amino acid sequence selected from any one of SEQ ID NOS: 1-295. In some embodiments, an antimicrobial composition comprises at least two peptides, each having an amino acid sequence selected from any one of SEQ ID NOS: 1, 6, 11, 16, 21, 26, 31, 36, 41, 76, 106, 142, 177, 178, 213, 218, 219, 220, 255, 256, and 291. In some embodiments, the antimicrobial composition of at least two peptides as disclosed herein, may provide for prolonged antimicrobial activity.

For contemplated compositions having antimicrobial activity, the composition comprises a peptide selected from any one of SEQ ID NOS: 1-295, the peptide formulated with a solvent and a viscosity enhancing agent. Non-limiting examples of a suitable solvent include glycerin, ethanol, methanol, propylene glycol, and/or isopropanol. In exemplary embodiments, the antimicrobial composition comprises a solvent selected from glycerin, propylene glycol, or isopropanol in a concentration ranging from 0.1 to 30% by volume; or the antimicrobial composition comprises a solvent selected from methanol or ethanol at a concentration up to 99% by volume.

In exemplary embodiments, an antimicrobial composition comprises a peptide having an amino acid sequence selected from an amino acid sequence selected from any one of SEQ ID NOS: 1, 6, 11, 16, 21, 26, 31, 36, 41, 76, 106, 142, 177, 178, 213, 218, 219, 220, 255, 256, and 291.

A method of inhibiting microbial growth or treating a microbial infection in a cell or a subject, comprises administering a composition comprising a peptide selected from any one of SEQ ID NOS: 1-295 to the infected cell or subject. The antimicrobial peptide composition may be formulated with a solvent and a viscosity enhancing agent. The microbe may be a bacterial, fungal (e.g., yeast), or viral pathogen.

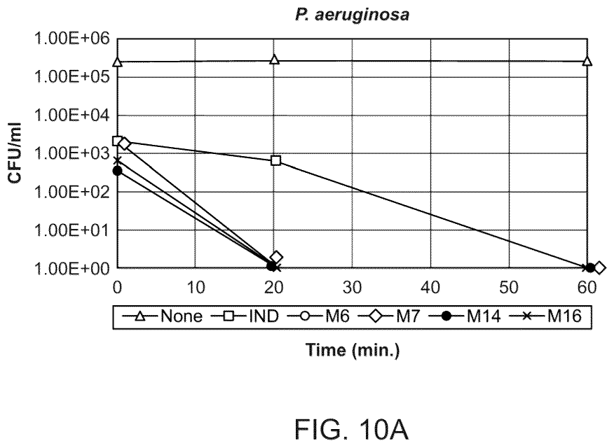

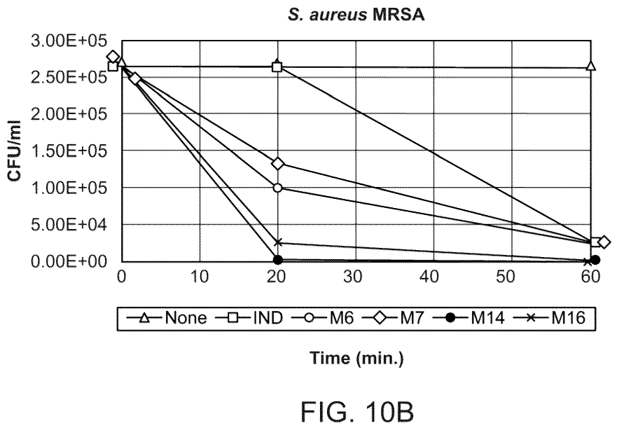

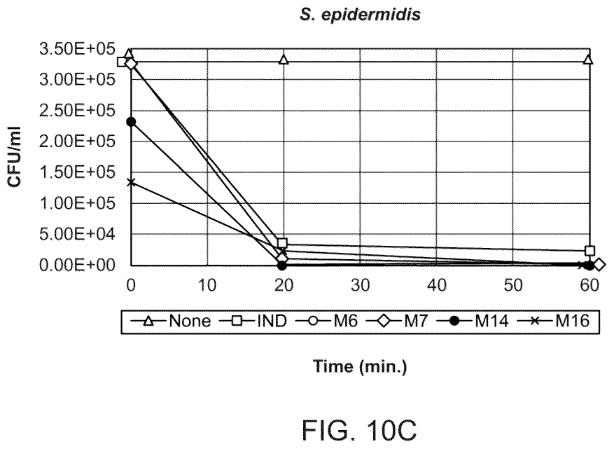

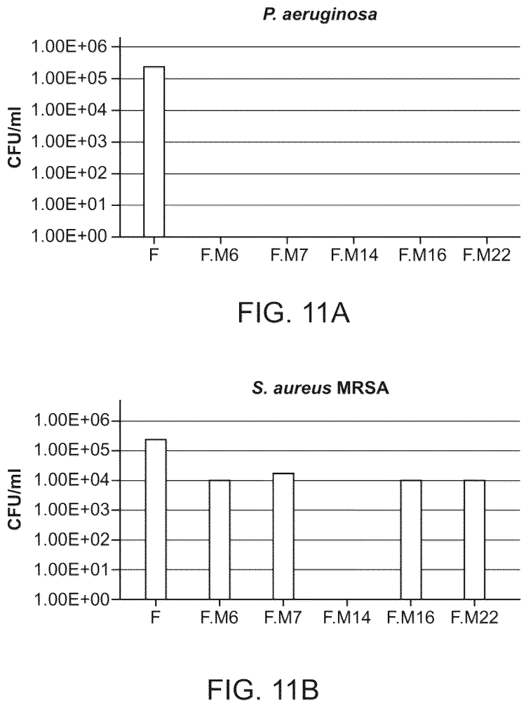

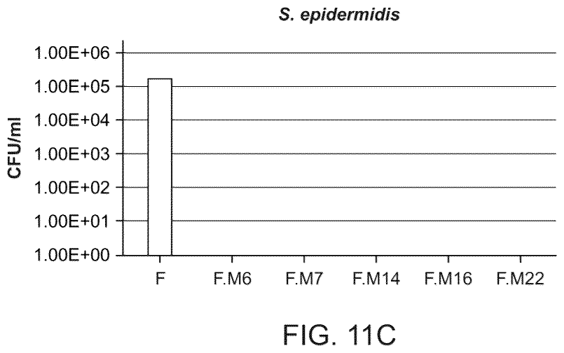

With reference to FIGS. 10A-10C and 11A-11C, a peptide or peptide composition as disclosed herein is administered in a method of inhibiting microbial growth or treating a microbial infection of a bacterial pathogen such as P. aeruginosa, S. aureus, or S. epidermis. A method of inhibiting growth of, or treating an infection of, P. aeruginosa or S. epidermis, comprises administering an antimicrobial peptide selected from M6 (SEQ ID NO: 26), M7 (SEQ ID NO: 31), M14 (SEQ ID NO: 177), and M16 (SEQ ID NO: 213) to the infected cell or subject. A method of inhibiting growth or treating a S. aureus infection comprises administering M14 peptide (SEQ ID NO: 177) or a composition comprising the M14 peptide (SEQ ID NO: 177).

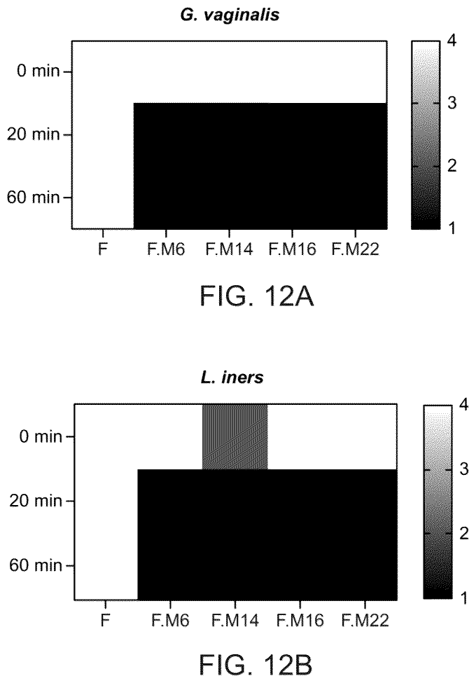

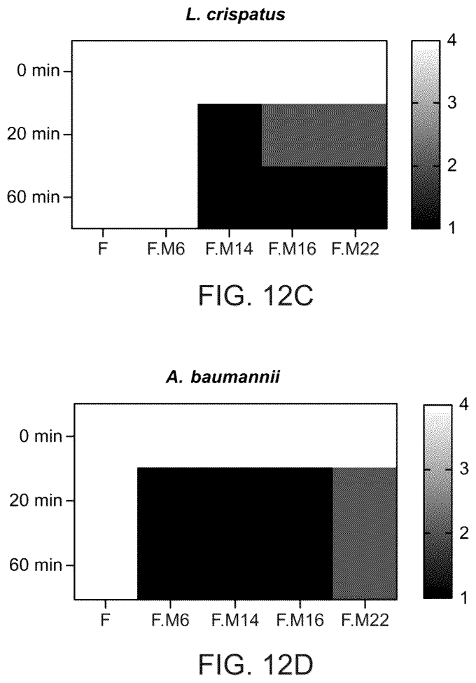

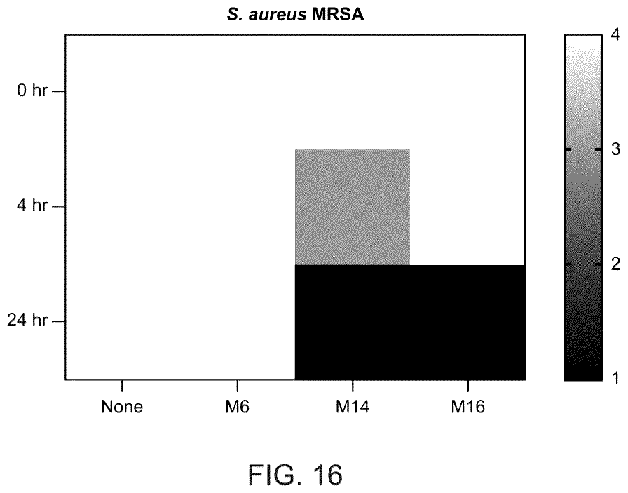

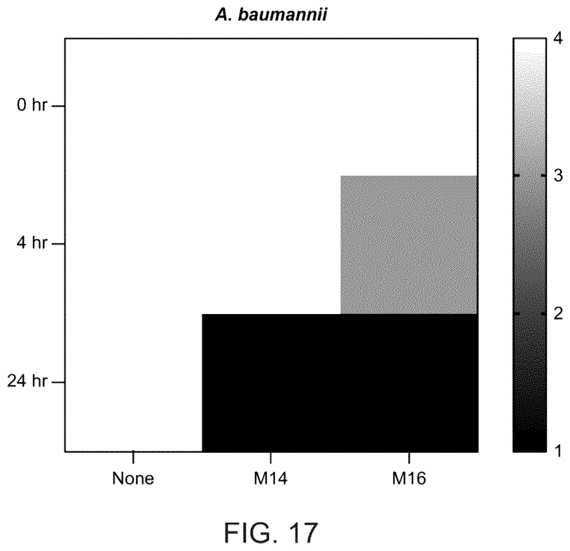

With reference to FIGS. 12A-12D and FIG. 17, a peptide or peptide composition as disclosed herein is administered in a method of inhibiting microbial growth or treating a microbial infection of a bacterial pathogen such as G. Vaginalis, L. iners, or A. baumannii. A method of inhibiting growth of, or treating an infection of, G. Vaginalis, L. iners, or A. baumannii comprises administering an antimicrobial peptide selected from M6 (SEQ ID NO: 26), M14 (SEQ ID NO:177), M16 (SEQ ID NO: 213) and M22 (SEQ ID NO: 291). Advantageously, L. crispatus is not susceptible to M6 (SEQ ID NO: 26) (FIG. 12C). Additionally, growth of A. baumannii is more sensitive to M16 (SEQ ID NO: 213) as compared to M22 (SEQ ID NO: 291) (FIG. 12D). Accordingly, a method of inhibiting growth or treating an infection of G. Vaginalis, L. iners, or A. baumannii, comprises administering a peptide or peptide composition of M6 (SEQ ID NO: 26), M14 (SEQ ID NO:177), M16 (SEQ ID NO: 213) or M22 (SEQ ID NO: 291), where administering M6 peptide or peptide composition would not inhibit L. crispatus, and administering M16 peptide or peptide composition more effectively treats A. baumannii.

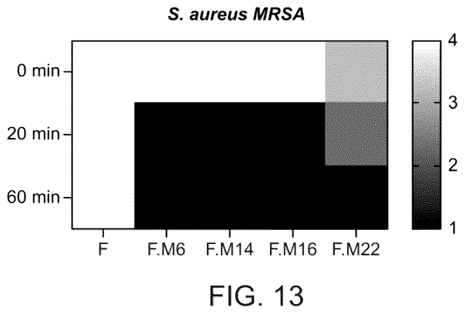

With reference to FIG. 13, a peptide composition comprising a peptide of M6 (SEQ ID NO: 26), M14 (SEQ ID NO:177), M16 (SEQ ID NO: 213) or M22 (SEQ ID NO: 291) is formulated with a solvent and viscosity enhancing agent to inhibit growth of or treat an infection of S. aureus MRSA. The peptide concentration in the formulation may range from 0.10 to 1.0% by weight. The addition of a solvent and viscosity enhancing agent as disclosed herein may increase the antimicrobial activity of the peptide composition. For example, the peptide composition may comprise a solvent of 20% propylene glycol and 2% hydroxypropyl cellulose.

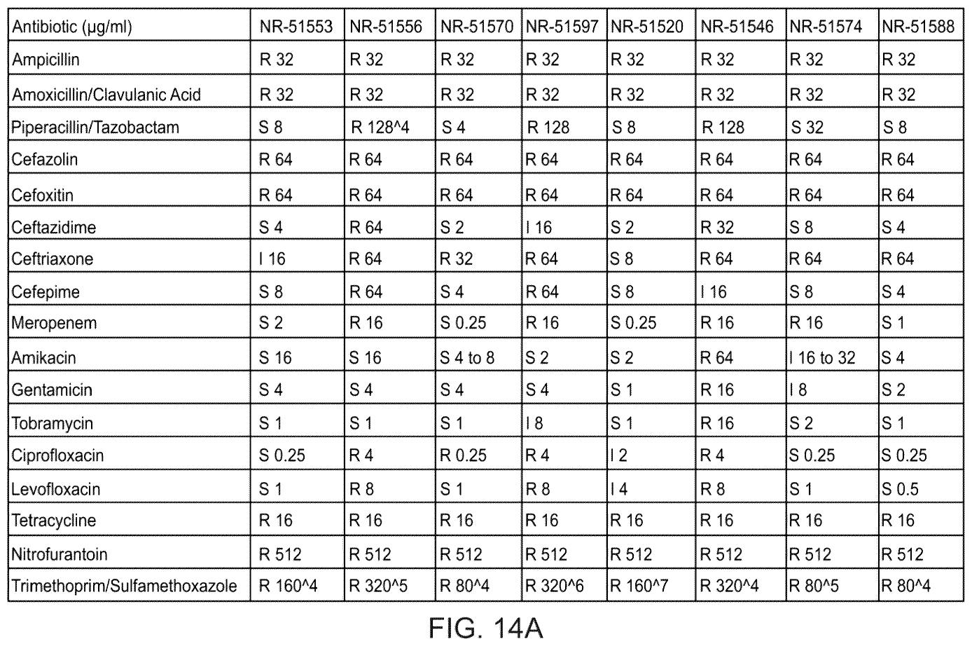

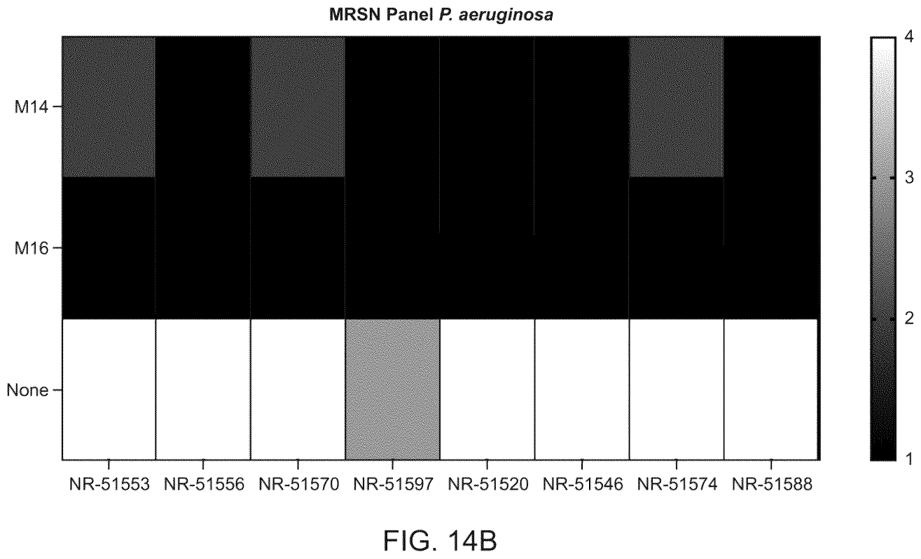

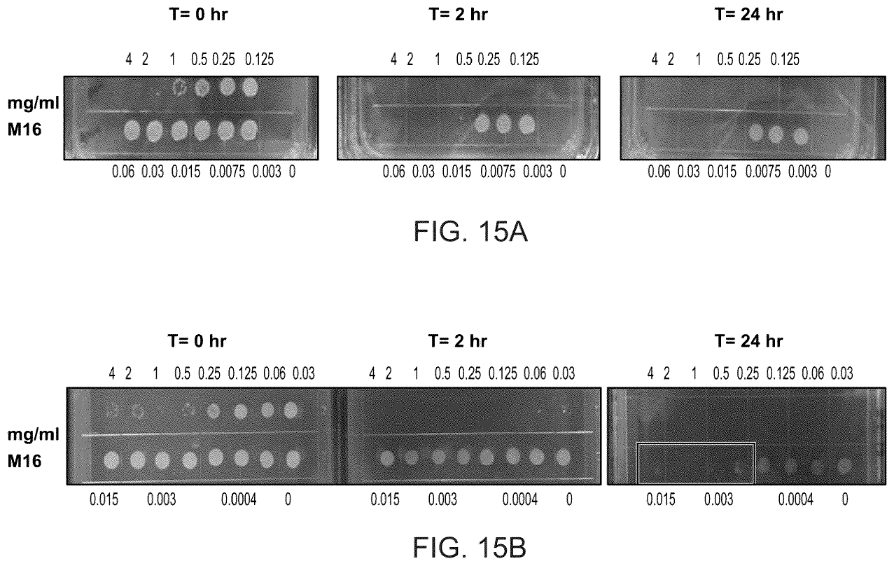

With reference to FIGS. 14A, 14B, 15A, and 15B, a peptide or peptide composition as disclosed herein is administered in a method of inhibiting microbial growth or treating a microbial infection of a bacterial pathogen such as strains of P. aeruginosa. A method of inhibiting growth of, or treating an infection of P. aeruginosa comprises administering an antimicrobial peptide selected from M14 (SEQ ID NO:177) and M16 (SEQ ID NO: 213). A peptide composition of M14 (SEQ ID NO:177) or M16 (SEQ ID NO: 213) for treating P. aeruginosa is formulated with a viscosity increasing agent as disclosed herein, to increase microbial activity of the composition. For example, with reference to FIGS. 14B and 15B, a peptide composition of M14 (SEQ ID NO:177) or M16 (SEQ ID NO: 213) is formulated with carboxymethyl cellulose (CMC).

In some embodiments, the peptides and polypeptides are synthesized using chemical synthesis from amino acids using synthesizers, or via use of expression systems including but not limited to bacteria, yeast, insect or mammalian systems. The peptide may also be generated by enzymatic cleavage or any suitable method for chromatographic separations.

For purification and/or isolation, the peptide may be fused to any suitable tag. Examples of suitable tags include, but are not limited to MBP, HIS6, protein A, protein G, GST, HSA, or fluorescent tags. Additionally or alternatively, the peptide may be fused with a cleavage site.

Administration of an antimicrobial peptide composition to a human or non-human animal or to a surface to inhibit or prevent microbial growth therein or thereon, may be accomplished with selected formulation of the antimicrobial peptide composition. Accordingly, the peptide composition may comprise one or more peptides in salt form or as an ester of the peptide as disclosed herein. The peptide composition disclosed herein may comprise an acid selected from a salt, a free acid, or any ester.

Administration to human and non-human animals can be accomplished using formulations that comprise solvent(s), acid(s), the one or more peptides in salt form, esters of the one or more peptides, buffering agent(s), viscosity enhancing agent(s), stabilizers, and humectants.

The contemplated compositions disclosed herein may include a stabilizer. In exemplary embodiments, the stabilizer is selected from one or more of carboxy methyl cellulose, pectin, guar gum, and gelatin.

The compositions disclosed herein may include a viscosity enhancing agent. For example, a viscosity enhancing agent is one or more independently selected from hydroxypropyl cellulose, hydroxypropyl methylcellulose, carboxymethyl cellulose (CMC), polyvinylpyrrolidone, dextran, and hyaluronic acid. In an exemplary embodiment, the viscosity enhancing agent is provided at concentrations ranging from about 0.4% to about 6% by volume of the composition.

The contemplated compositions disclosed herein may include one or more organic acids selected from citric acid, malic acid, tartaric acid, acetic acid, butyric acid, phosphoric acid, lactic acid, indole lactic acid, and/or indole ethanol. In some additional embodiments, the antimicrobial composition includes an additional salt in combination with the one or more organic acids for specific pH formulations.

The contemplated compositions disclosed herein may include N or C-terminal modifications, PEGylation, glycolsylation, and/or other suitable synthetic modifications thereof.

The contemplated compositions disclosed herein may include a humectant. In some embodiments, the humectant is one or more selected from sorbitol, glycerol, propylene glycol, sorbitol, seaweed, urea, peptides, amino acids, and honey. In some embodiments, the contemplated compositions disclosed herein may include a humectant. In some embodiments, the humectant is sorbitol or glycerol. In some embodiments, the antimicrobial composition includes a humectant together with one or more preservatives independently selected from benzoic acid, benzyl alcohol, propylparaben, methyl paraben, and phenoxyethanol.

The peptide and peptide compositions as disclosed herein, may be formulated as a spray, gel, ointment, wash, cream, or insert tablet (for vaginal delivery). In some embodiments, the antimicrobial composition includes a solvent. In exemplary embodiments, each solvent is independently selected from glycerin, ethanol, methanol, propylene glycol, and isopropanol. In exemplary embodiments, the antimicrobial composition includes one or more solvent independently selected from glycerin, propylene glycol, and isopropanol in a concentration ranging from about 0.1 to about 30% based on the total weight or volume of the composition; or the antimicrobial composition includes a solvent selected from methanol and ethanol at a concentration up to about 99% based on the total weight or volume of the composition.

The contemplated compositions disclosed herein may include an acid selected from a salt, a free acid, or any ester.

The contemplated compositions disclosed herein may include a buffering agent. In exemplary embodiments, the buffering agent is added in a range of about 1 to about 300 mM. In exemplary embodiments, the buffering agent is one or more independently selected from acetate, succinate, lactate, fumarate or any additional monocarboxylates and dicarboxylates.

In some embodiments, a method of treating a microbial infection comprises administering to a subject suffering from a microbial infection, a composition comprising one or more peptides having an amino acid sequence selected from any one of SEQ ID NOS: 1-295. In exemplary embodiments, a method of treating a microbial infection comprises administering a composition comprising one or more peptides having an amino acid sequence selected from any one of SEQ ID NOS: 1-140, 142-217, 220-254, and/or 256-295. In exemplary embodiments, a method of treating a microbial infection comprises administering a composition comprising one or more peptides having an amino acid sequence selected from any one of M6 (SEQ ID NO: 26), M7 (SEQ ID NO: 31), M14 (SEQ ID NO: 177), M16 (SEQ ID NO: 213), and M22 (SEQ ID NO: 291). A microbial infection comprises a bacterial infection, a fungal infection, and/or a viral infection. In exemplary embodiments, a microbial infection comprises a bacterial infection or a viral infection.

In certain embodiments, the administration is via cell harboring the polynucleotide sequence that would encode the polypeptide sequence for delivery for human or non-human application.

Antimicrobial compositions disclosed herein also include cosmetic compositions comprising one or more of the peptides as disclosed herein. For example, the antimicrobial composition as disclosed herein may be combined with or formulated as a cosmetic composition. In some embodiments, the combination of the contemplated antimicrobial composition (e.g., one or more peptides represented by SEQ ID NOS: 1-295) with any suitable cosmetic composition prolongs the stability and shelf life of the cosmetic composition compared to the cosmetic composition without the antimicrobial composition. The type of cosmetic composition is not limited and may include creams, lotions, cleansers, solutions (e.g., toners, water), serums, ointments, and make-up (blush, powder, eyeliner, and mascara).

Additionally, a topical skin composition for improving skin health may comprise one or more peptides as disclosed herein. The addition of an antimicrobial peptide (e.g., having bactericidal and/or antifungal activity) to the skin reduces inflammation, improves wound healing, and/or enhances the integrity of the skin barrier. Accordingly, a topical skin composition for improving skin health comprises one or more peptides independently selected from SEQ ID NOS: 1-295. A topical skin composition for enhancing skin health may comprise a peptide as disclosed herein, in concentration from about 0.1 mg/ml to 50 mg/ml of peptide. The topical skin composition may comprise a peptide concentration in a range selected from: about 0.1 mg/ml to 50 mg/ml; about 1 mg/ml to about 50 mg/ml; about 2 mg/ml to about 45 mg/ml; about 5 mg/ml to about 40 mg/ml; about 5 mg/ml to about 30 mg/ml; about 5 mg/ml to about 25 mg/ml; about 5 mg/ml to about 20 mg/ml; about 5 mg/ml to about 15 mg/ml; and about 10 mg/ml to about 30 mg/ml. The topical skin composition may comprise about 0.25 mg/ml to about 10 mg/ml; about 0.25 to about 5 mg/ml; about 0.50 mg/ml to about 10 mg/ml; about 1 to about 10 mg/ml; about 2 to about 10 mg/ml; about 3 to about 10 mg/ml; about 4 to about 10 mg/ml; about 5 mg/ml to about 10 mg/ml; about 6 mg/ml to about 10 mg/ml; about 7 mg/ml to about 10 mg/ml; about 8 mg/ml to about 10 mg/ml; about 9 mg/ml to about 10 mg/ml; about 0.50 mg/ml to about 5 mg/ml; about 1 mg/ml to about 8 mg/ml; about 2 mg/ml to about 8 mg/ml; and about 2 mg/ml to about 6 mg/ml. Specifically, a topical skin composition comprises a peptide selected from M6 (SEQ ID NO: 26), M14 (SEQ ID NO: 177), M16 (SEQ ID NO: 213), and M22 (SEQ ID NO: 291). A method of enhancing skin health includes administering to the skin of a subject (e.g., human or animal) a topical composition comprising a peptide selected from M6 (SEQ ID NO: 26), M14 (SEQ ID NO: 177), M16 (SEQ ID NO: 213), and M22 (SEQ ID NO: 291) having a peptide concentration in a range as disclosed herein.

### M1-M22 and Variant Peptides (SEO ID NOS: 1-295)

TABLE 1A
M1-M2 Peptide Variants

| | |
|---|---|
| | M1 |
| SEQ ID NO: 1 | PKVERRPRPR |
| SEQ ID NO: 2 | PKVERRPRP |
| SEQ ID NO: 3 | PKVERRPR |
| SEQ ID NO: 4 | KVERRPRPR |
| SEQ ID NO: 5 | VERRPRPR |
| | M2 |
| SEQ ID NO: 6 | GKRKPVTRAR |
| SEQ ID NO: 7 | GKRKPVTRA |
| SEQ ID NO: 8 | GKRKPVTR |
| SEQ ID NO: 9 | KRKPVTRAR |
| SEQ ID NO: 10 | RKPVTRAR |

TABLE 1B
M3-M4 Peptide Variants

| | |
|---|---|
| | M3 |
| SEQ ID NO: 11 | QHQKAKKPWT |
| SEQ ID NO: 12 | QHQKAKKPW |
| SEQ ID NO: 13 | QHQKAKKP |
| SEQ ID NO: 14 | HQKAKKPWT |
| SEQ ID NO: 15 | QKAKKPWT |
| | M4 |
| SEQ ID NO: 16 | HQKMAKPWTQ |
| SEQ ID NO: 17 | HQKMAKPWT |
| SEQ ID NO: 18 | HQKMAKPW |
| SEQ ID NO: 19 | QKMAKPWTQ |
| SEQ ID NO: 20 | KMAKPWTQ |

TABLE 1C
M5-M6 Peptide Variants

| | |
|---|---|
| | M5 |
| SEQ ID NO: 21 | NTNRPRTKRA |
| SEQ ID NO: 22 | NTNRPRTKR |
| SEQ ID NO: 23 | NTNRPRTK |
| SEQ ID NO: 24 | TNRPRTKRA |
| SEQ ID NO: 25 | NRPRTKRA |
| | M6 |
| SEQ ID NO: 26 | RPRIPKASAK |
| SEQ ID NO: 27 | RPRIPKASA |
| SEQ ID NO: 28 | RPRIPKAS |
| SEQ ID NO: 29 | PRIPKASAK |
| SEQ ID NO: 30 | RIPKASAK |

TABLE 1D
M7-M8 Peptide Variants

| | |
|---|---|
| | M7 |
| SEQ ID NO: 31 | PVPQKVRPQR |
| SEQ ID NO: 32 | PVPQKVRPQ |
| SEQ ID NO: 33 | PVPQKVRP |
| SEQ ID NO: 34 | VPQKVRPQR |
| SEQ ID NO: 35 | PQKVRPQR |
| | M8 |
| SEQ ID NO: 36 | KTPKAVNKCR |
| SEQ ID NO: 37 | KTPKAVNKC |
| SEQ ID NO: 38 | KTPKAVNK |
| SEQ ID NO: 39 | TPKAVNKCR |
| SEQ ID NO: 40 | PKAVNKCR |

TABLE 1E
M9 Peptide Variants

| | M9 |
|---|---|
| SEQ ID NO: 41 | ATKCKQWQRNMRKVR |
| SEQ ID NO: 42 | ATKCKQWQRNMRKV |
| SEQ ID NO: 43 | ATKCKQWQRNMRK |
| SEQ ID NO: 44 | ATKCKQWQRNMR |
| SEQ ID NO: 45 | ATKCKQWQRNM |
| SEQ ID NO: 46 | ATKCKQWQRN |
| SEQ ID NO: 47 | ATKCKQWQR |
| SEQ ID NO: 48 | ATKCKQWQ |
| SEQ ID NO: 49 | TKCKQWQRNMRKVR |
| SEQ ID NO: 50 | TKCKQWQRNMRKV |
| SEQ ID NO: 51 | TKCKQWQRNMRK |
| SEQ ID NO: 52 | TKCKQWQRNMR |
| SEQ ID NO: 53 | TKCKQWQRNM |
| SEQ ID NO: 54 | TKCKQWQRN |
| SEQ ID NO: 55 | TKCKQWQR |
| SEQ ID NO: 56 | KCKQWQRNMRKVR |
| SEQ ID NO: 57 | KCKQWQRNMRKV |
| SEQ ID NO: 58 | KCKQWQRNMRK |
| SEQ ID NO: 59 | KCKQWQRNMR |
| SEQ ID NO: 60 | KCKQWQRNM |
| SEQ ID NO: 61 | KCKQWQRN |
| SEQ ID NO: 62 | CKQWQRNMRKVR |
| SEQ ID NO: 63 | CKQWQRNMRKV |
| SEQ ID NO: 64 | CKQWQRNMRK |
| SEQ ID NO: 65 | CKQWQRNMR |
| SEQ ID NO: 66 | CKQWQRNM |
| SEQ ID NO: 67 | KQWQRNMRKVR |
| SEQ ID NO: 68 | KQWQRNMRKV |
| SEQ ID NO: 69 | KQWQRNMRK |
| SEQ ID NO: 70 | KQWQRNMR |
| SEQ ID NO: 71 | QWQRNMRKVR |
| SEQ ID NO: 72 | QWQRNMRKV |
| SEQ ID NO: 73 | QWQRNMRK |
| SEQ ID NO: 74 | WQRNMRKVR |
| SEQ ID NO: 75 | WQRNMRKV |

TABLE 1F
M10 Peptide Variants

| | M10 |
|---|---|
| SEQ ID NO: 76 | KTKCFQWQRNMRKVR |
| SEQ ID NO: 77 | KTKCFQWQRNMRKV |
| SEQ ID NO: 78 | KTKCFQWQRNMRK |
| SEQ ID NO: 79 | KTKCFQWQRNMR |
| SEQ ID NO: 80 | KTKCFQWQRNM |
| SEQ ID NO: 81 | KTKCFQWQRN |
| SEQ ID NO: 82 | KTKCFQWQR |
| SEQ ID NO: 83 | KTKCFQWQ |
| SEQ ID NO: 84 | TKCFQWQRNMRKVR |
| SEQ ID NO: 85 | TKCFQWQRNMRKV |
| SEQ ID NO: 86 | TKCFQWQRNMRK |
| SEQ ID NO: 87 | TKCFQWQRNMR |
| SEQ ID NO: 88 | TKCFQWQRNM |
| SEQ ID NO: 89 | TKCFQWQRN |
| SEQ ID NO: 90 | TKCFQWQR |
| SEQ ID NO: 91 | KCFQWQRNMRKVR |
| SEQ ID NO: 92 | KCFQWQRNMRKV |
| SEQ ID NO: 93 | KCFQWQRNMRK |
| SEQ ID NO: 94 | KCFQWQRNMR |
| SEQ ID NO: 95 | KCFQWQRNM |
| SEQ ID NO: 96 | KCFQWQRN |
| SEQ ID NO: 97 | CFQWQRNMRKVR |
| SEQ ID NO: 98 | CFQWQRNMRKV |
| SEQ ID NO: 99 | CFQWQRNMRK |
| SEQ ID NO: 100 | CFQWQRNMR |
| SEQ ID NO: 101 | CFQWQRNM |
| SEQ ID NO: 102 | FQWQRNMRKVR |
| SEQ ID NO: 103 | FQWQRNMRKV |
| SEQ ID NO: 104 | FQWQRNMRK |
| SEQ ID NO: 105 | FQWQRNMR |

TABLE 1G
M11 Peptide Variants

| | M11 |
|---|---|
| SEQ ID NO: 106 | KTKCKQRQRNMRKVR |
| SEQ ID NO: 107 | KTKCKQRQRNMRKV |
| SEQ ID NO: 108 | KTKCKQRQRNMRK |
| SEQ ID NO: 109 | KTKCKQRQRNMR |
| SEQ ID NO: 110 | KTKCKQRQRNM |
| SEQ ID NO: 111 | KTKCKQRQRN |
| SEQ ID NO: 112 | KTKCKQRQR |
| SEQ ID NO: 113 | KTKCKQRQ |
| SEQ ID NO: 114 | TKCKQRQRNMRKVR |
| SEQ ID NO: 115 | TKCKQRQRNMRKV |
| SEQ ID NO: 116 | TKCKQRQRNMRK |
| SEQ ID NO: 117 | TKCKQRQRNMR |
| SEQ ID NO: 118 | TKCKQRQRNM |
| SEQ ID NO: 119 | TKCKQRQRN |
| SEQ ID NO: 120 | TKCKQRQR |
| SEQ ID NO: 121 | KCKQRQRNMRKVR |
| SEQ ID NO: 122 | KCKQRQRNMRKV |
| SEQ ID NO: 123 | KCKQRQRNMRK |
| SEQ ID NO: 124 | KCKQRQRNMR |
| SEQ ID NO: 125 | KCKQRQRNM |
| SEQ ID NO: 126 | KCKQRQRN |
| SEQ ID NO: 127 | CKQRQRNMRKVR |
| SEQ ID NO: 128 | CKQRQRNMRKV |
| SEQ ID NO: 129 | CKQRQRNMRK |
| SEQ ID NO: 130 | CKQRQRNMR |
| SEQ ID NO: 131 | CKQRQRNM |
| SEQ ID NO: 132 | KQRQRNMRKVR |
| SEQ ID NO: 133 | KQRQRNMRKV |
| SEQ ID NO: 134 | KQRQRNMRK |
| SEQ ID NO: 135 | KQRQRNMR |
| SEQ ID NO: 136 | QRQRNMRKVR |
| SEQ ID NO: 137 | QRQRNMRKV |
| SEQ ID NO: 138 | QRQRNMRK |
| SEQ ID NO: 139 | RQRNMRKVR |
| SEQ ID NO: 140 | RQRNMRKV |

TABLE 1H
M12, M13, and M14 Peptide Variants

| | |
|---|---|
| | M12 |
| SEQ ID NO: 141 | KQRQRNMRKVR |
| | M13 |
| SEQ ID NO: 142 | MRKVRGPPKSCRKRD |
| SEQ ID NO: 143 | MRKVRGPPKSCRKR |
| SEQ ID NO: 144 | MRKVRGPPKSCRK |
| SEQ ID NO: 145 | MRKVRGPPKSCR |
| SEQ ID NO: 146 | MRKVRGPPKSC |
| SEQ ID NO: 147 | MRKVRGPPKS |
| SEQ ID NO: 148 | MRKVRGPPK |
| SEQ ID NO: 149 | MRKVRGPP |
| SEQ ID NO: 150 | RKVRGPPKSCRKRD |
| SEQ ID NO: 151 | RKVRGPPKSCRKR |
| SEQ ID NO: 152 | RKVRGPPKSCRK |
| SEQ ID NO: 153 | RKVRGPPKSCR |
| SEQ ID NO: 154 | RKVRGPPKSC |
| SEQ ID NO: 155 | RKVRGPPKS |
| SEQ ID NO: 156 | RKVRGPPK |
| SEQ ID NO: 157 | KVRGPPKSCRKRD |
| SEQ ID NO: 158 | KVRGPPKSCRKR |
| SEQ ID NO: 159 | KVRGPPKSCRK |
| SEQ ID NO: 160 | KVRGPPKSCR |
| SEQ ID NO: 161 | KVRGPPKSC |
| SEQ ID NO: 162 | KVRGPPKS |
| SEQ ID NO: 163 | VRGPPKSCRKRD |
| SEQ ID NO: 164 | VRGPPKSCRKR |
| SEQ ID NO: 165 | VRGPPKSCRK |
| SEQ ID NO: 166 | VRGPPKSCR |
| SEQ ID NO: 167 | VRGPPKSC |
| SEQ ID NO: 168 | RGPPKSCRKRD |
| SEQ ID NO: 169 | RGPPKSCRKR |
| SEQ ID NO: 170 | RGPPKSCRK |
| SEQ ID NO: 171 | RGPPKSCR |
| SEQ ID NO: 172 | GPPKSCRKRD |
| SEQ ID NO: 173 | GPPKSCRKR |
| SEQ ID NO: 174 | GPPKSCRK |
| SEQ ID NO: 175 | PPKSCRKRD |
| SEQ ID NO: 176 | PPKSCRKR |
| | M14 |
| SEQ ID NO: 177 | RGPPKSCRKRD |

TABLE 1I
M15 Peptide Variants

| | M15 |
|---|---|
| SEQ ID NO: 178 | RQPRTHKYAVRVVKK |
| SEQ ID NO: 179 | RQPRTHKYAVRVVK |
| SEQ ID NO: 180 | RQPRTHKYAVRVV |
| SEQ ID NO: 181 | RQPRTHKYAVRV |
| SEQ ID NO: 182 | RQPRTHKYAVR |
| SEQ ID NO: 183 | RQPRTHKYAV |
| SEQ ID NO: 184 | RQPRTHKYA |
| SEQ ID NO: 185 | RQPRTHKY |
| SEQ ID NO: 186 | QPRTHKYAVRVVKK |
| SEQ ID NO: 187 | QPRTHKYAVRVVK |
| SEQ ID NO: 188 | QPRTHKYAVRVV |
| SEQ ID NO: 189 | QPRTHKYAVRV |
| SEQ ID NO: 190 | QPRTHKYAVR |
| SEQ ID NO: 191 | QPRTHKYAV |
| SEQ ID NO: 192 | QPRTHKYA |
| SEQ ID NO: 193 | PRTHKYAVRVVKK |
| SEQ ID NO: 194 | PRTHKYAVRVVK |
| SEQ ID NO: 195 | PRTHKYAVRVV |
| SEQ ID NO: 196 | PRTHKYAVRV |
| SEQ ID NO: 197 | PRTHKYAVR |
| SEQ ID NO: 198 | PRTHKYAV |
| SEQ ID NO: 199 | RTHKYAVRVVKK |
| SEQ ID NO: 200 | RTHKYAVRVVK |
| SEQ ID NO: 201 | RTHKYAVRVV |
| SEQ ID NO: 202 | RTHKYAVRV |
| SEQ ID NO: 203 | RTHKYAVR |
| SEQ ID NO: 204 | THKYAVRVVKK |
| SEQ ID NO: 205 | THKYAVRVVK |
| SEQ ID NO: 206 | THKYAVRVV |
| SEQ ID NO: 207 | THKYAVRV |
| SEQ ID NO: 208 | HKYAVRVVKK |
| SEQ ID NO: 209 | HKYAVRVVK |
| SEQ ID NO: 210 | HKYAVRVV |
| SEQ ID NO: 211 | KYAVRVVKK |
| SEQ ID NO: 212 | KYAVRVVK |

TABLE 1J
M16, M17, M18, M20,
M22 Peptide Variants

| | |
|---|---|
| | M16 |
| SEQ ID NO: 213 | HKYAVRVVKK |
| SEQ ID NO: 214 | HKYAVRVVK |
| SEQ ID NO: 215 | HKYAVRVV |
| SEQ ID NO: 216 | KYAVRVVKK |
| SEQ ID NO: 217 | KYAVRVVK |
| | M17 |
| SEQ ID NO: 218 | ATKCFQWQRNMRKVR |
| | M18 |
| SEQ ID NO: 219 | FQWQRNMRKVR |
| | M19 |
| SEQ ID NOS: 220-254 | Table 1K |
| | M20 |
| SEQ ID NO: 255 | RGPPVSCIKRD |
| | M21 |
| SEQ ID NOS: 256-290 | Table 1L |
| | M22 |
| SEQ ID NO: 291 | HYYAVAVVK |
| SEQ ID NO: 292 | HYYAVAVV |
| SEQ ID NO: 293 | HYYAVAV |
| SEQ ID NO: 294 | YYAVAVVKK |
| SEQ ID NO: 295 | YYAVAVVK |

TABLE 1K
M19 Peptide Variants

| | M19 |
|---|---|
| SEQ ID NO: 220 | MRKVRGPPVSCIKRD |
| SEQ ID NO: 221 | MRKVRGPPVSCIKR |
| SEQ ID NO: 222 | MRKVRGPPVSCIK |
| SEQ ID NO: 223 | MRKVRGPPVSCI |
| SEQ ID NO: 224 | MRKVRGPPVSC |
| SEQ ID NO: 225 | MRKVRGPPVS |
| SEQ ID NO: 226 | MRKVRGPPV |
| SEQ ID NO: 227 | MRKVRGPP |
| SEQ ID NO: 228 | RKVRGPPVSCIKRD |
| SEQ ID NO: 229 | RKVRGPPVSCIKR |
| SEQ ID NO: 230 | RKVRGPPVSCIK |
| SEQ ID NO: 231 | RKVRGPPVSCI |
| SEQ ID NO: 232 | RKVRGPPVSC |
| SEQ ID NO: 233 | RKVRGPPVS |
| SEQ ID NO: 234 | RKVRGPPV |
| SEQ ID NO: 235 | KVRGPPVSCIKRD |
| SEQ ID NO: 236 | KVRGPPVSCIKR |
| SEQ ID NO: 237 | KVRGPPVSCIK |
| SEQ ID NO: 238 | KVRGPPVSCI |
| SEQ ID NO: 239 | KVRGPPVSC |
| SEQ ID NO: 240 | KVRGPPVS |
| SEQ ID NO: 241 | VRGPPVSCIKRD |
| SEQ ID NO: 242 | VRGPPVSCIKR |
| SEQ ID NO: 243 | VRGPPVSCIK |
| SEQ ID NO: 244 | VRGPPVSCI |
| SEQ ID NO: 245 | VRGPPVSC |
| SEQ ID NO: 246 | RGPPVSCIKRD |
| SEQ ID NO: 247 | RGPPVSCIKR |
| SEQ ID NO: 248 | RGPPVSCIK |
| SEQ ID NO: 249 | RGPPVSCI |
| SEQ ID NO: 250 | GPPVSCIKRD |
| SEQ ID NO: 251 | GPPVSCIKR |
| SEQ ID NO: 252 | GPPVSCIK |
| SEQ ID NO: 253 | PPVSCIKRD |
| SEQ ID NO: 254 | PPVSCIKR |

TABLE 1L
M21 Peptide Variants

| | M21 |
|---|---|
| SEQ ID NO: 256 | RQPRTHYYAVAVVKK |
| SEQ ID NO: 257 | RQPRTHYYAVAVVK |
| SEQ ID NO: 258 | RQPRTHYYAVAVV |
| SEQ ID NO: 259 | RQPRTHYYAVAV |
| SEQ ID NO: 260 | RQPRTHYYAVA |
| SEQ ID NO: 261 | RQPRTHYYAV |
| SEQ ID NO: 262 | RQPRTHYYA |
| SEQ ID NO: 263 | RQPRTHYY |
| SEQ ID NO: 264 | QPRTHYYAVAVVKK |
| SEQ ID NO: 265 | QPRTHYYAVAVVK |
| SEQ ID NO: 266 | QPRTHYYAVAVV |
| SEQ ID NO: 267 | QPRTHYYAVAV |
| SEQ ID NO: 268 | QPRTHYYAVA |
| SEQ ID NO: 269 | QPRTHYYAV |
| SEQ ID NO: 270 | QPRTHYYA |
| SEQ ID NO: 271 | PRTHYYAVAVVKK |
| SEQ ID NO: 272 | PRTHYYAVAVVK |
| SEQ ID NO: 273 | PRTHYYAVAVV |
| SEQ ID NO: 274 | PRTHYYAVAV |
| SEQ ID NO: 275 | PRTHYYAVA |
| SEQ ID NO: 276 | PRTHYYAV |
| SEQ ID NO: 277 | RTHYYAVAVVKK |
| SEQ ID NO: 278 | RTHYYAVAVVK |
| SEQ ID NO: 279 | RTHYYAVAVV |
| SEQ ID NO: 280 | RTHYYAVAV |
| SEQ ID NO: 281 | RTHYYAVA |
| SEQ ID NO: 282 | THYYAVAVVKK |
| SEQ ID NO: 283 | THYYAVAVVK |
| SEQ ID NO: 284 | THYYAVAVV |
| SEQ ID NO: 285 | THYYAVAV |
| SEQ ID NO: 286 | HYYAVAVVKK |
| (same as SEQ ID | |
| NO: 291) | |
| SEQ ID NO: 287 | HYYAVAVVK |
| (same as SEQ ID | |
| NO: 292) | |
| SEQ ID NO: 288 | HYYAVAVV |
| (same as SEQ ID | |
| NO: 293) | |
| SEQ ID NO: 289 | YYAVAVVKK |
| (same as SEQ ID | |
| NO: 294) | |
| SEQ ID NO: 290 | YYAVAVVK |
| (same as SEQ ID | |
| NO: 295) | |

### EXAMPLES

Synthetic peptides M1-M16 and natural peptides M17-M22 correspond to SEQ ID NOS: 1, 6, 11, 16, 21, 26, 31, 36, 41, 76, 106, 141, 142, 177, 178, 213, 218, 219, 220, 255, 256, or 291, respectively, and are examples of some of the peptides of mammalian milk proteins disclosed herein and listed in FIG. 7. All of peptides M1-M22 are derived from or are from camel K-casein (FIG. 1), human Lactotransferrin (FIG. 2), goat S2-casein (FIG. 3), human Lactase-phlorizin hydrolase (FIG. 4), goat β-casein (FIG. 5), and bovine Lysozyme C isozyme (FIG. 6).

As set forth in Tables 1A to 1L, the peptides disclosed herein are between 8-15 amino acids in length and cationic in nature—i.e., the peptides are positively charged with charge ranging from 2.1 to up to 7.9.

For certain sequences, the natural sequences could not be synthesized, so only modified synthetic peptides were analyzed. For example, the synthesis of the natural sequence from which M14 (SEQ ID NO: 177) was derived failed. Similarly, the synthesis of the natural sequence from which M9 (SEQ ID NO: 41) M10 (SEQ ID NO: 76), and M11 (SEQ ID NO: 106) were derived, failed. Peptides were purchased from Genescript, Inc.

M1-M8 (SEQ ID NOS: 1, 6, 11, 16, 21, 26, 31, and 36) were tested using direct kill assays. Equal inoculums of G. vaginalis, L. iners and L. crispatus were incubated with either water (0.00), or 0.375 mg/ml, 0.75 mg/ml and 1.5 mg/ml. of peptides. Aliquots were spotted 30 min post incubation on HBT-bilayer media, Tryptic soy agar (+5% sheep blood) or MRS agar plates respectively. For these peptides, 0.375 mg/ml concentration is sufficient to cause loss of viability of G. vaginalis and L. iners as compared to the absence of peptides.

In FIG. 8A, M1-M8 peptides (SEQ ID NOS: 1, 6, 11, 16, 21, 26, 31, and 36) were tested using direct kill assays. Equal inoculums of G. vaginalis, L. iners and L. crispatus were incubated with either water (0.00), or 0.375 mg/ml, 0.75 mg/ml and 1.5 mg/ml. of peptides. Aliquots were spotted 30 minutes (min) post incubation on human blood tween (HBT) bilayer media, Tryptic soy agar (+5% sheep blood) or MRS (de Man, Rogosa, and Sharpe) agar plates respectively. For these peptides, 0.375 mg/ml concentration is sufficient to cause loss of viability of G. vaginalis and L. iners as compared to the absence of peptides. The relative minimal bactericidal concentration (MBC) for each of M1-M8 peptides is provided in FIG. 8B.

In FIG. 9, the bacterial pathogens P. aeruginosa and S. aureus—associated with skin, burn and wound infections were tested. As shown, P. aeruginosa is more sensitive to the peptides compared to S. aureus, and certain peptides are more effective than others when tested in time-kill assays. For example, M6-M8 (SEQ ID NOS: 26, 31, and 36), M14 (SEQ ID NO: 177) and M15 (SEQ ID NO: 178) are more effective in reducing the viability of P. aeruginosa as compared to M1-M5 (SEQ ID NOS: 1, 6, 11, 16, and 21).

In FIGS. 10A, 10B, and 10C, quantitative colony-forming units/ml were assayed using the indicated peptides at a final concentration of 0.5% (5 mg/ml). For this colony-forming assay, peptides were incubated with indicated bacterial strains in 0.8% NaCl, and aliquots were removed starting time T=0, up to 60 minutes, followed by serial dilution and plating on Tryptic soy agar plates for overnight incubation, and enumeration of CFU/ml. It was observed that P. aeruginosa is particularly sensitive to peptides M6 (SEQ ID NO: 26), M7 (SEQ ID NO: 31), M14 (SEQ ID NO: 177), and M16 (SEQ ID NO: 213). Similarly, S. aureus and S. epidermidis are also sensitive to these peptides. Specifically, both P. aeruginosa and S. aureus MRSA appear more sensitive to these peptides as compared to another cationic peptide Indolicidin after 20 minutes, as indicated.

In FIGS. 11A, 11B, and 11C, the impact of the formulation comprising 20% propylene glycol and 2% hydroxypropyl cellulose and 0.5% by weight of peptides M6 (SEQ ID NO: 26), M7 (SEQ ID NO: 31), M14 (SEQ ID NO: 177), M16 (SEQ ID NO: 213), or M22 (SEQ ID NO: 291) was tested in a colony-forming assay. While the formulation on its own (F) (without peptide) does not alter the viability of cells, the presence of the peptides at 0.5% final concentration in the formulation continues to permit antibacterial activity of select peptides. P. aeruginosa and S. epidermidis remain more susceptible to activity as compared to S. aureus.

In FIGS. 12A, 12B, 12C, and 12D, the impact of the formulation comprising 20% propylene glycol and 2% hydroxypropyl cellulose and 0.5% by weight of peptide M6 (SEQ ID NO: 26), M7 (SEQ ID NO: 31), M14 (SEQ ID NO: 177), M16 (SEQ ID NO: 213), or M22 (SEQ ID NO: 291) was tested in a direct kill assay as described herein. While the formulation on its own (F) (without peptide) does not alter the viability of cells, the presence of the peptides in the formulation retains antibacterial activity of these peptides—e.g., P. aeruginosa and S. epidermidis remain more susceptible to activity as compared to S. aureus.

In FIG. 13, the impact of the formulation comprising 20% propylene glycol and 2% hydroxypropyl cellulose and 0.5% by weight of peptide M6 (SEQ ID NO: 26), M7 (SEQ ID NO: 31), M14 (SEQ ID NO: 177), M16 (SEQ ID NO: 213), or M22 (SEQ ID NO: 291) was tested using a direct kill assay. While the formulation on its own (F) (without peptide) does not alter the viability of S. aureus MRSA, the presence of the peptides in the formulation retains antibacterial activity of these peptides, with the observation of M22 (SEQ ID NO: 291) reducing viability starting at time 0 min.

FIG. 14A indicates sensitive(S) or resistant (R) relative to the listed antibiotic for eight of the more peptide-resistant S. aureus strains of 100 MRSN panel strains (NR-51553, NR-51556, NR-51570, NR51597, NR-51520, NR-51546, NR-51574, and NR-51588). FIG. 14B shows the impact of the formulation comprising 0.030 mg/ml (30 μg/ml) peptide M14 (SEQ ID NO: 177) and M16 (SEQ ID NO: 213) in a Clinical and Laboratory Standards Institute (CLSI)-based broth dilution method to determine the minimal bactericidal concentration (MBC) for M14 and M16 in the presence of 0.003% (30 μg/ml) peptide for each of the eight tested MRSN S. aureus strains, as indicated. CLSI-based method described in CLSI document M07-A10, Vol. 35, No. 2, 2015 (ISBN 1-56238-988-2).

FIGS. 15A and 15B show results of a modified CLSI-based dilution method as described above, to test peptide M16 (SEQ ID NO: 213) on MRSN panel P. aeruginosa strain NR-51553 in the absence of carboxymethyl cellulose and 0.01×MH (Mueller Hinton) broth (FIG. 15B) and the presence of 0.5% CMC and 0.01×MH broth (FIG. 15B), resulting in a minimal bactericidal concentration (MBC) of 15 μg/ml for this more resistant P. aeruginosa strains.

FIG. 16. shows results of a modified CLSI-based dilution method as described above, to test peptide M6 (SEQ ID NO: 26), M14 (SEQ ID NO: 177), and M16 (SEQ ID NO: 213) on S. aureus MRSA, in which 0.01×MH broth at 250 μg/ml M14 (SEQ ID NO: 177) is more efficacious at an earlier time (T) of 4 hours, compared to peptide of M16 (SEQ ID NO: 213) at 24 hours.

FIG. 17. shows results of a direct kill assay of A. baumannii, in which 250 g/ml M16 (SEQ ID NO: 213) is more efficacious at an earlier time (T) of 4 hours, compared to peptide of M14 (SEQ ID NO: 177) at 24 hours.

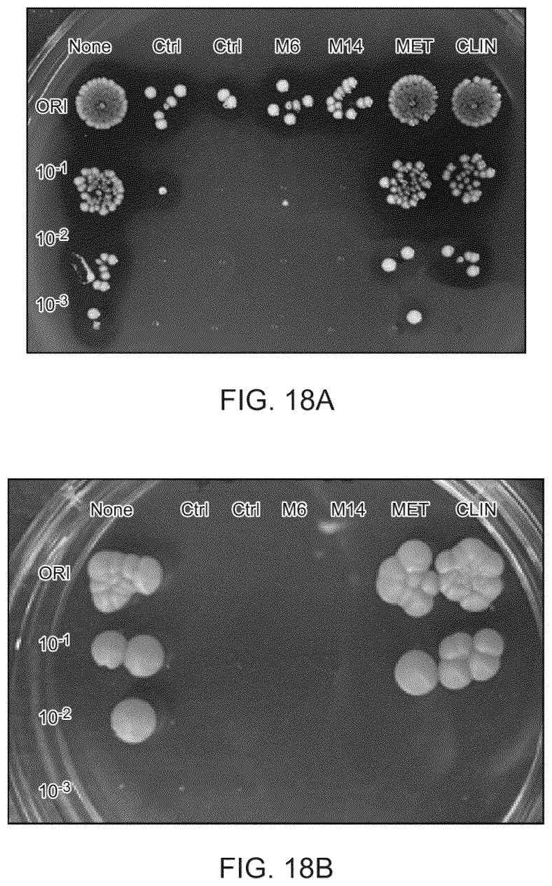

FIG. 18A. shows results of an ex vivo assay of bactericidal activity using vaginal swabs from patients with symptoms for bacterial vaginosis in a direct kill assay as disclosed herein, to test M6 (SEQ ID NO: 26) and M14 (SEQ ID NO: 177) side by side with metronidazole (MET) and clindamycin (CLIN) in the presence of the bacteria E. faecalis on HBT bilary agar. FIG. 18B shows results of this ex vivo assay of activity using vaginal swabs from patients to test M6 (SEQ ID NO: 26) and M14 (SEQ ID NO: 177) side by side with metronidazole (MET) and clindamycin (CLIN) in the presence of the Candida albicans on MRS agar.

### Cytoxicity Assay

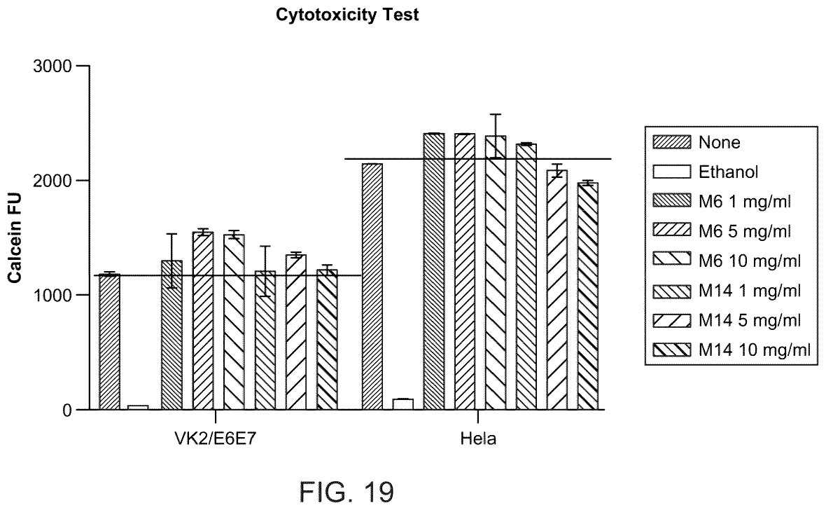

FIG. 19 shows the cytoxicity results of peptides M6 (SEQ ID NO: 26) and M14 (SEQ ID NO: 177) assayed on vaginal epithelial cells (VK2/E6E7) and cervical cells (HeLa) using a standard calcein dye cytoxicity assay (e.g., Thermofisher (Invitrogen), Catalog Number L3224). The peptides were incubated with the cells for 18 hours at 1 mg/ml, 5 mg/ml, and 10 mg/ml peptide concentrations, with a no peptide (None) control and an Ethanol positive control, as indicated. The M6 and M14 peptides retained viability up to 20 mg/ml.

### Biofilm Assay

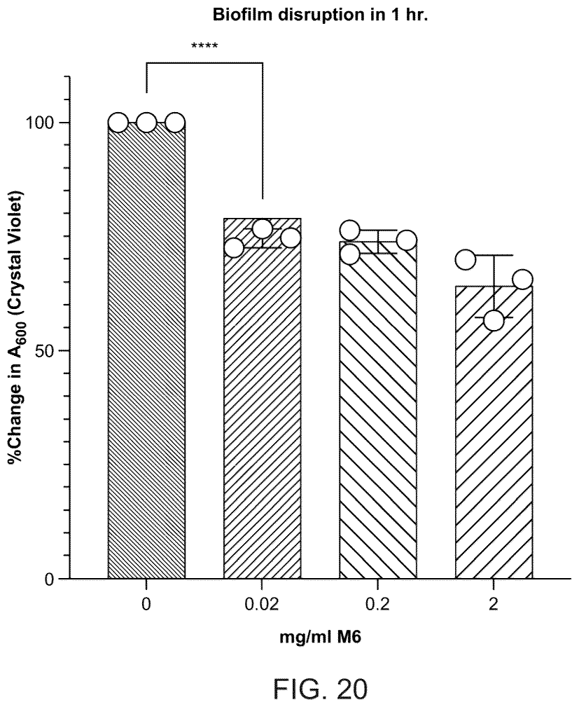

FIG. 20 shows the results of the peptide M6 (SEQ ID NO: 26) in a G. vaginalis biofilm assay the measures disruption of the biofilm with a decrease in crystal violet dye as described, for example, in O'Toole, 2011, J. Vis. Exp., 47:2437. In brief, the disruption of the G. vaginalis biofilm was measured with increasing concentrations of M6 peptide, at 0.02 mg/ml, 0.2 mg/ml, and 2 mg/ml M6 peptide the crystal violet dye was reduced, indicating biofilm disruption of G. vaginalis.

### EpiVaginal Model

Table 2 below show the results of peptides M6 (SEQ ID NO: 26) and M14 (SEQ ID NO: 177) were assayed for their impact on vaginal irritation using the Epi Vaginal model to determine the exposure time to decrease the tissue viability to 50% (ET50) in EpiVaginal™ tissue model (Mattek). In brief, after exposure to the M6 and M14 peptides at 20 mg/ml final concentration for various times, toxicity was determined by measuring the relative conversion of MTT (3-(4,5-dimethylthiazol-2-yl)-2,5-dephenyltetrazolium bromide) in the vaginal tissue samples treated with peptide compared to tissues treated with a solvent negative control. Positive control was Triton-X-100 at 1% final concentration.

TABLE 2

| Test article | Peptide Concentration | ET₅₀ (hours) |
|---|---|---|
| M6 | 2% | >24 |
| M14 | 2% | >24 |
| Triton-X-100 | 1% | 1.55 |

### In Vivo Assays

In vivo antimicrobial activity of 0.5% (5 mg/ml) M6 (SEQ ID NO: 26), M14 (SEQ ID NO: 177), M16 (SEQ ID NO: 213) and/or M22 (SEQ ID NO: 291) peptide in 0.5% carboxymethyl cellulose (CMC) is carried out in two models: 1) a custom excisional wound infection model with P. aeruginosa strain PAO1 (ATCC15691) where the efficacy of the antimicrobial peptide is compared to one control drug, (e.g., ciprofloxacin); and 2) an established dermal infection model for S. aureus, USA300 strain of MRSA, (ATCC® BAA-1717™) where the efficacy of the antimicrobial peptide (AMP) is compared to commercially available mupirocin ointment (e.g., Bactroban® or Centany®), as well as A. baumannii infection models. For these in vivo models, mice are infected with the relevant pathogen (e.g., P. aeruginosa, S. aureus, A. baumannii). Control mice are sacrificed at 1-hour post pathogen infection and mice tested with positive control or peptide receive twice daily (BID) dosing at 12-hour intervals over a period of 2.5 days. At end of each relevant timepoint (e.g., at 2.5 days), the mice are sacrificed and bacterial counts are enumerated from the wound area at 11 hours after the last dose.

### Antiviral Assays

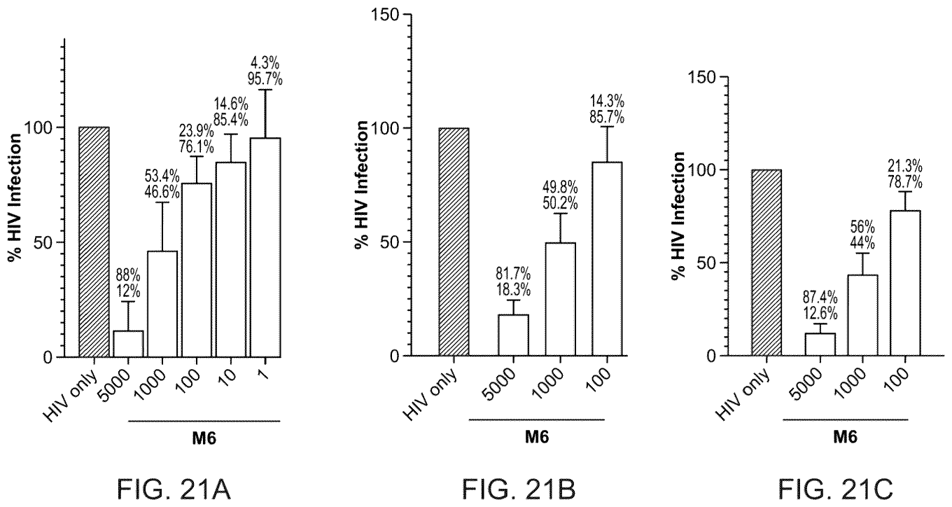

Inhibition of the human immunodeficiency virus (HIV) was assayed using TZM-bl cells exposed to peptide M6 (SEQ ID NO: 26) at concentrations of 5,000, 1,000, 100, 10, or 1 μg/ml, with or without HIV-1 for 48 hours, in addition to controls. In FIG. 21A, cells were exposed to only HIV or only media for a co-exposure experiment. In a pre-exposure setting (FIG. 21B), the cells were exposed to media or peptide M6 (SEQ ID NO: 26) at the indicated concentrations (μg/ml) for 6 hours (6h), then washed, and subsequently exposed to HIV for 36 to 42 hours. In a post-exposure experiment (FIG. 21C), the cells are exposed to HIV for 2 hours, washed, and then exposed to HIV and peptide M6 (SEQ ID NO: 26) at the indicated concentrations for 11 hours, washed, and incubated with media for 36 hours. For the evaluation of inhibition, a bright-glo luciferase assay system (e.g., Britelite, PerkinElmer or Brite-Glo, Promega) as described, e.g., in Sarzotti-Kelsoe et al., 2014, J. Immunol. Methods, 0:131-146 or Puertas et al., 2012, J. Clin. Microbiol., 50:3909-3916, was used to determine the percentage of HIV infection where cells were exposed to virus and peptides compared to control wells where cells are exposed to virus only. The entire contents of Sarzotti-Kelsoe et al., 2014 and Puertas et al., 2012 are herein incorporated by reference.

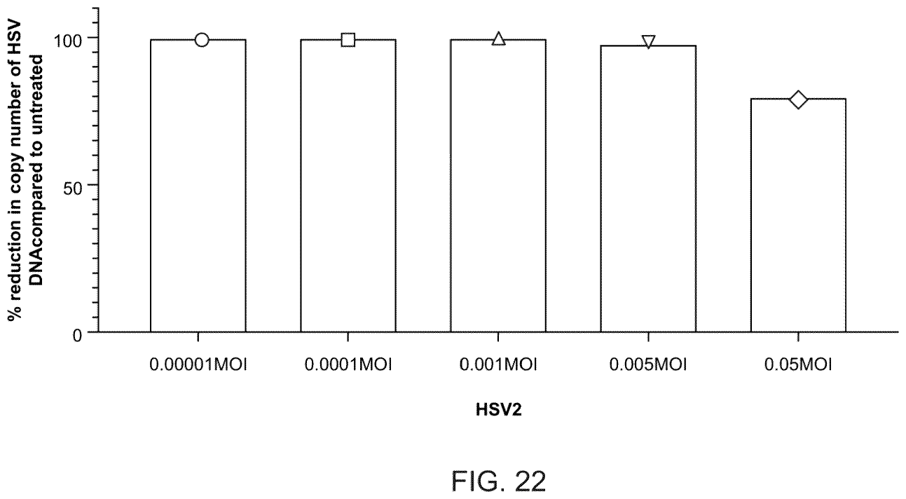

In FIG. 22, inhibition of herpes simplex virus (HSV) was tested using HEC1A cells infected with HSV-2G virus at specific multiplicity of infection (MOI) as indicated. In brief, after 1 hour of exposure, the virus is removed and peptide M6 (SEQ ID NO: 26) was added at 5 mg/ml for 3 days prior to qRT-PCR to assay for HSV-2 DNA in cell culture supernatants.

1. A peptide having an amino acid sequence selected from any one of SEQ ID NOS: 1-295; or a pharmaceutically or cosmetically acceptable salt thereof.

2. A composition comprising the peptide of claim 1, or a pharmaceutically or cosmetically acceptable salt thereof, further comprising a viscosity increasing agent.

3. The composition of claim 2, wherein the viscosity increasing agent is selected from the group consisting of hydroxypropyl cellulose, hydroxypropyl methylcellulose, carboxymethyl cellulose (CMC), polyvinylpyrrolidone, dextran, and hyaluronic acid.

4. The composition of claim 2 or 3, wherein the viscosity increasing agent is hydroxypropyl cellulose or carboxymethyl cellulose (CMC).

5. The composition of any one of claims 2-4, wherein the viscosity increasing agent is provided at a concentration ranging from about 0.4% to about 6% based on the total weight or volume of the composition.

6. The composition of any one of claims 2-5, further comprising a solvent.

7. The composition of claim 6, wherein the solvent is selected from one or more of the group consisting of glycerin, ethanol, methanol, propylene glycol, and isopropanol.

8. The composition of claim 6, wherein the solvent is selected from one or more of glycerin, propylene glycol, and isopropanol at a concentration ranging from 0.1 to 30%; or the solvent is selected from methanol and ethanol at a concentration up to 99%.

9. The composition of any one of claims 6-8, wherein the solvent is propylene glycol.

10. The peptide of claim 1 or the composition of any one of claims 2-9, wherein the peptide, or a pharmaceutically or cosmetically acceptable salt thereof, has an amino acid sequence selected from SEQ ID NOS: 1, 6, 11, 16, 21, 26, 31, 36, 41, 76, 106, 141, 142, 177, 178, 213, 218, 219, 220, 255, 256, and 291.

11. The peptide of claim 1 or the composition of any one of claims 2-9, wherein the peptide, or a pharmaceutically or cosmetically acceptable salt thereof, has an amino acid sequence selected from SEQ ID NOS: 1, 6, 11, 16, 21, 26, 31, 36, 41, 76, 106, 141, 142, 177, 178, 213, 219, 220, 256, and 291.

12. The peptide of claim 1 or the composition of any one of claims 2-9, wherein the peptide, or a pharmaceutically or cosmetically acceptable salt thereof, has an amino acid sequence selected from any one of SEQ ID NOS: 26, 177, 178, 213, and 291.

13. The composition of any one of claims 2-12, wherein the peptide is present in an amount from about 0.02 mg/ml to 50 mg/mL.

14. The composition of any one of claims 2-13, wherein the peptide is present in an amount from about 0.02 mg/mL to 20 mg/mL.

15. The composition of any one of claims 2-14, wherein the peptide is present an amount from about 0.375 mg/mL to 10 mg/mL.

16. A method of treating a microbial infection in a subject suffering from a microbial infection, the method comprising administering the peptide or the composition of any one of claims 1-15 to the subject.

17. The method of claim 16, wherein the microbial infection is selected from a bacterial, fungal, and a viral infection.

18. The method of claim 16, wherein the microbial infection is a bacterial infection selected from P. aeruginosa, S. aureus, S. epidermidis, G. vaginalis, L. iners, A. baumannii, and E. faecalis infections.

19. The method of claim 16, wherein the microbial infection is a C. albicans yeast infection.

20. The method of claim 16, wherein the microbial infection is a viral infection selected from HIV and herpes simplex virus (HSV) infections.

21. A method of treating a surface comprising a microbe, the method comprising administering the peptide or the composition of any one of claims 1-15 to the surface.

22. The method of claim 21, wherein the surface is a medical device.

23. A composition comprising a first peptide having an amino acid sequence selected from any one of SEQ ID NOS: 1-295 or a pharmaceutically or cosmetically acceptable salt thereof; and a second peptide having an amino acid sequence selected from any one of SEQ ID NOS: 1-295 or a pharmaceutically or cosmetically acceptable salt thereof, wherein the first peptide and the second peptide are different.

24. The composition of claim 23, further comprising a viscosity increasing agent.

25. The composition of claim 24, wherein the viscosity increasing agent is selected from the group consisting of hydroxypropyl cellulose, hydroxypropyl methylcellulose, carboxymethyl cellulose (CMC), polyvinylpyrrolidone, dextran, and hyaluronic acid.

26. The composition of claim 24 or 25, wherein the viscosity increasing agent is provided at a concentration ranging from 0.4% to 6% based on the total weight or volume of the composition.

27. The composition of any of claims 23-26, further comprising a solvent.

28. The composition of claim 27, wherein the solvent is selected from the group consisting of glycerin, ethanol, methanol, propylene glycol, and isopropanol.

29. The composition of claim 27, wherein the solvent is selected from glycerin, propylene glycol, dimethylsulfoxide (DMSO), and isopropanol at a concentration ranging from 0.1 to 30%; or the solvent is selected from methanol and ethanol at a concentration up to 99%.

30. The composition of any one of claims 23-29, wherein the first peptide and the second peptide are selected from any one of SEQ ID NOS: 1, 6, 11, 16, 21, 26, 31, 36, 41, 76, 106, 141, 142, 177, 178, 213, 218, 219, 220, 255, 256, and 291 and a pharmaceutically or cosmetically acceptable salt thereof.

31. The composition of any one of claims 23-29, wherein the first peptide is selected from any one of SEQ ID NOS: 1, 6, 11, 16, 21, 26, 31, 36, 41, 76, 106, 141, 142, 177, 178, 213, 218, 219, 220, 255, 256, and 291 and the second peptide is selected from any one of SEQ ID NOS: 26, 31, 36, 177, 178, and 213 and a pharmaceutically or cosmetically acceptable salt thereof.

32. The composition of any one of claims 23-29, wherein the first peptide and the second peptide are selected from any one of SEQ ID NOS: 26, 177, 213, and 291 and a pharmaceutically or cosmetically acceptable salt thereof.

33. The composition of any one of claims 23-32, further comprising one or more additional peptides selected from the group consisting of 1-295.

34. A method of treating a microbial infection in a subject suffering from a microbial infection, the method comprising administering the composition of any one of claims 23-33 to the subject.

35. The method of claim 34, wherein the microbial infection is selected from a bacterial, fungal and viral infection.

36. The method of claim 34, wherein the microbial infection is a bacterial infection selected from P. aeruginosa, S. aureus, S. epidermidis, G. vaginalis, L. iners, A. baumannii, and E. faecalis.

37. A method of treating a surface comprising a microbe, the method comprising administering the composition of any one of claims 23-33 to the surface.

38. The method of claim 37, wherein the surface is a medical device.

39. A cosmetic composition comprising the peptide or the composition of any one of claim 1-15 or 23-33, wherein the peptide is optionally a pharmaceutically or cosmetically acceptable salt thereof.

40. The cosmetic composition of claim 39, wherein the cosmetic composition is selected from a cream, lotion, cleanser, solution, serum, ointment, and make-up.

41. The cosmetic composition of claim 39 or 40, wherein the cosmetic composition has increased shelf life compared to the cosmetic composition without the peptide composition.


SEQUENCE LISTING (301 sequences)

SEQ ID NO: 1 - protein, 10 aa, synthetic construct
  REGION 1..10: note = Synthetic: M1
PKVERRPRPR

SEQ ID NO: 2 - protein, 9 aa, synthetic construct
  REGION 1..9: note = Synthetic: M1
PKVERRPRP

SEQ ID NO: 3 - protein, 8 aa, synthetic construct
  REGION 1..8: note = Synthetic: M1
PKVERRPR

SEQ ID NO: 4 - protein, 9 aa, synthetic construct
  REGION 1..9: note = Synthetic: M1
KVERRPRPR

SEQ ID NO: 5 - protein, 8 aa, synthetic construct
  REGION 1..8: note = Synthetic: M1
VERRPRPR

SEQ ID NO: 6 - protein, 10 aa, synthetic construct
  REGION 1..10: note = Synthetic: M2
GKRKPVTRAR

SEQ ID NO: 7 - protein, 9 aa, synthetic construct
  REGION 1..9: note = Synthetic: M2
GKRKPVTRA

SEQ ID NO: 8 - protein, 8 aa, synthetic construct
  REGION 1..8: note = Synthetic: M2
GKRKPVTR

SEQ ID NO: 9 - protein, 9 aa, synthetic construct
  REGION 1..9: note = Synthetic: M2
KRKPVTRAR

SEQ ID NO: 10 - protein, 8 aa, synthetic construct
  REGION 1..8: note = Synthetic: M2
RKPVTRAR

SEQ ID NO: 11 - protein, 10 aa, synthetic construct
  REGION 1..10: note = Synthetic: M3
QHQKAKKPWT

SEQ ID NO: 12 - protein, 9 aa, synthetic construct
  REGION 1..9: note = Synthetic: M3
QHQKAKKPW

SEQ ID NO: 13 - protein, 8 aa, synthetic construct
  REGION 1..8: note = Synthetic: M3
QHQKAKKP

SEQ ID NO: 14 - protein, 9 aa, synthetic construct
  REGION 1..9: note = Synthetic: M3
HQKAKKPWT

SEQ ID NO: 15 - protein, 8 aa, synthetic construct
  REGION 1..8: note = Synthetic: M3
QKAKKPWT

SEQ ID NO: 16 - protein, 10 aa, synthetic construct
  REGION 1..10: note = Synthetic: M4
HQKMAKPWTQ

SEQ ID NO: 17 - protein, 9 aa, synthetic construct
  REGION 1..9: note = Synthetic: M4
HQKMAKPWT

SEQ ID NO: 18 - protein, 8 aa, synthetic construct
  REGION 1..8: note = Synthetic: M4
HQKMAKPW

SEQ ID NO: 19 - protein, 9 aa, synthetic construct
  REGION 1..9: note = Synthetic: M4
QKMAKPWTQ

SEQ ID NO: 20 - protein, 8 aa, synthetic construct
  REGION 1..8: note = Synthetic: M4
KMAKPWTQ

SEQ ID NO: 21 - protein, 10 aa, synthetic construct
  REGION 1..10: note = Synthetic: M5
NTNRPRTKRA

SEQ ID NO: 22 - protein, 9 aa, synthetic construct
  REGION 1..9: note = Synthetic: M5
NTNRPRTKR

SEQ ID NO: 23 - protein, 8 aa, synthetic construct
  REGION 1..8: note = Synthetic: M5
NTNRPRTK

SEQ ID NO: 24 - protein, 9 aa, synthetic construct
  REGION 1..9: note = Synthetic: M5
TNRPRTKRA

SEQ ID NO: 25 - protein, 8 aa, synthetic construct
  REGION 1..8: note = Synthetic: M5
NRPRTKRA

SEQ ID NO: 26 - protein, 10 aa, synthetic construct
  REGION 1..10: note = Synthetic: M6
RPRIPKASAK

SEQ ID NO: 27 - protein, 9 aa, synthetic construct
  REGION 1..9: note = Synthetic: M6
RPRIPKASA

SEQ ID NO: 28 - protein, 8 aa, synthetic construct
  REGION 1..8: note = Synthetic: M6
RPRIPKAS

SEQ ID NO: 29 - protein, 9 aa, synthetic construct
  REGION 1..9: note = Synthetic: M6
PRIPKASAK

SEQ ID NO: 30 - protein, 8 aa, synthetic construct
  REGION 1..8: note = Synthetic: M6
RIPKASAK

SEQ ID NO: 31 - protein, 10 aa, synthetic construct
  REGION 1..10: note = Synthetic: M7
PVPQKVRPQR

SEQ ID NO: 32 - protein, 9 aa, synthetic construct
  REGION 1..9: note = Synthetic: M7
PVPQKVRPQ

SEQ ID NO: 33 - protein, 8 aa, synthetic construct
  REGION 1..8: note = Synthetic: M7
PVPQKVRP

SEQ ID NO: 34 - protein, 9 aa, synthetic construct
  REGION 1..9: note = Synthetic: M7
VPQKVRPQR

SEQ ID NO: 35 - protein, 8 aa, synthetic construct
  REGION 1..8: note = Synthetic: M7
PQKVRPQR

SEQ ID NO: 36 - protein, 10 aa, synthetic construct
  REGION 1..10: note = Synthetic: M8
KTPKAVNKCR

SEQ ID NO: 37 - protein, 9 aa, synthetic construct
  REGION 1..9: note = Synthetic: M8
KTPKAVNKC

SEQ ID NO: 38 - protein, 8 aa, synthetic construct
  REGION 1..8: note = Synthetic: M8
KTPKAVNK

SEQ ID NO: 39 - protein, 9 aa, synthetic construct
  REGION 1..9: note = Synthetic: M8
TPKAVNKCR

SEQ ID NO: 40 - protein, 8 aa, synthetic construct
  REGION 1..8: note = Synthetic: M8
PKAVNKCR

SEQ ID NO: 41 - protein, 15 aa, synthetic construct
  REGION 1..15: note = Synthetic: M9
ATKCKQWQRNMRKVR

SEQ ID NO: 42 - protein, 14 aa, synthetic construct
  REGION 1..14: note = Synthetic: M9
ATKCKQWQRNMRKV

SEQ ID NO: 43 - protein, 13 aa, synthetic construct
  REGION 1..13: note = Synthetic: M9
ATKCKQWQRNMRK

SEQ ID NO: 44 - protein, 12 aa, synthetic construct
  REGION 1..12: note = Synthetic: M9
ATKCKQWQRNMR

SEQ ID NO: 45 - protein, 11 aa, synthetic construct
  REGION 1..11: note = Synthetic: M9
ATKCKQWQRNM

SEQ ID NO: 46 - protein, 10 aa, synthetic construct
  REGION 1..10: note = Synthetic: M9
ATKCKQWQRN

SEQ ID NO: 47 - protein, 9 aa, synthetic construct
  REGION 1..9: note = Synthetic: M9
ATKCKQWQR

SEQ ID NO: 48 - protein, 8 aa, synthetic construct
  REGION 1..8: note = Synthetic: M9
ATKCKQWQ

SEQ ID NO: 49 - protein, 14 aa, synthetic construct
  REGION 1..14: note = Synthetic: M9
TKCKQWQRNMRKVR

SEQ ID NO: 50 - protein, 13 aa, synthetic construct
  REGION 1..13: note = Synthetic: M9
TKCKQWQRNMRKV

SEQ ID NO: 51 - protein, 12 aa, synthetic construct
  REGION 1..12: note = Synthetic: M9
TKCKQWQRNMRK

SEQ ID NO: 52 - protein, 11 aa, synthetic construct
  REGION 1..11: note = Synthetic: M9
TKCKQWQRNMR

SEQ ID NO: 53 - protein, 10 aa, synthetic construct
  REGION 1..10: note = Synthetic: M9
TKCKQWQRNM

SEQ ID NO: 54 - protein, 9 aa, synthetic construct
  REGION 1..9: note = Synthetic: M9
TKCKQWQRN

SEQ ID NO: 55 - protein, 8 aa, synthetic construct
  REGION 1..8: note = Synthetic: M9
TKCKQWQR

SEQ ID NO: 56 - protein, 13 aa, synthetic construct
  REGION 1..13: note = Synthetic: M9
KCKQWQRNMRKVR

SEQ ID NO: 57 - protein, 12 aa, synthetic construct
  REGION 1..12: note = Synthetic: M9
KCKQWQRNMRKV

SEQ ID NO: 58 - protein, 11 aa, synthetic construct
  REGION 1..11: note = Synthetic: M9
KCKQWQRNMRK

SEQ ID NO: 59 - protein, 10 aa, synthetic construct
  REGION 1..10: note = Synthetic: M9
KCKQWQRNMR

SEQ ID NO: 60 - protein, 9 aa, synthetic construct
  REGION 1..9: note = Synthetic: M9
KCKQWQRNM

SEQ ID NO: 61 - protein, 8 aa, synthetic construct
  REGION 1..8: note = Synthetic: M9
KCKQWQRN

SEQ ID NO: 62 - protein, 12 aa, synthetic construct
  REGION 1..12: note = Synthetic: M9
CKQWQRNMRKVR

SEQ ID NO: 63 - protein, 11 aa, synthetic construct
  REGION 1..11: note = Synthetic: M9
CKQWQRNMRKV

SEQ ID NO: 64 - protein, 10 aa, synthetic construct
  REGION 1..10: note = Synthetic: M9
CKQWQRNMRK

SEQ ID NO: 65 - protein, 9 aa, synthetic construct
  REGION 1..9: note = Synthetic: M9
CKQWQRNMR

SEQ ID NO: 66 - protein, 8 aa, synthetic construct
  REGION 1..8: note = Synthetic: M9
CKQWQRNM

SEQ ID NO: 67 - protein, 11 aa, synthetic construct
  REGION 1..11: note = Synthetic: M9
KQWQRNMRKVR

SEQ ID NO: 68 - protein, 10 aa, synthetic construct
  REGION 1..10: note = Synthetic: M9
KQWQRNMRKV

SEQ ID NO: 69 - protein, 9 aa, synthetic construct
  REGION 1..9: note = Synthetic: M9
KQWQRNMRK

SEQ ID NO: 70 - protein, 8 aa, synthetic construct
  REGION 1..8: note = Synthetic: M9
KQWQRNMR

SEQ ID NO: 71 - protein, 10 aa, synthetic construct
  REGION 1..10: note = Synthetic: M9
QWQRNMRKVR

SEQ ID NO: 72 - protein, 9 aa, synthetic construct
  REGION 1..9: note = Synthetic: M9
QWQRNMRKV

SEQ ID NO: 73 - protein, 8 aa, synthetic construct
  REGION 1..8: note = Synthetic: M9
QWQRNMRK

SEQ ID NO: 74 - protein, 9 aa, synthetic construct
  REGION 1..9: note = Synthetic: M9
WQRNMRKVR

SEQ ID NO: 75 - protein, 8 aa, synthetic construct
  REGION 1..8: note = Synthetic: M9
WQRNMRKV

SEQ ID NO: 76 - protein, 15 aa, synthetic construct
  REGION 1..15: note = Synthetic: M10
KTKCFQWQRNMRKVR

SEQ ID NO: 77 - protein, 14 aa, synthetic construct
  REGION 1..14: note = Synthetic: M10
KTKCFQWQRNMRKV

SEQ ID NO: 78 - protein, 13 aa, synthetic construct
  REGION 1..13: note = Synthetic: M10
KTKCFQWQRNMRK

SEQ ID NO: 79 - protein, 12 aa, synthetic construct
  REGION 1..12: note = Synthetic: M10
KTKCFQWQRNMR

SEQ ID NO: 80 - protein, 11 aa, synthetic construct
  REGION 1..11: note = Synthetic: M10
KTKCFQWQRNM

SEQ ID NO: 81 - protein, 10 aa, synthetic construct
  REGION 1..10: note = Synthetic: M10
KTKCFQWQRN

SEQ ID NO: 82 - protein, 9 aa, synthetic construct
  REGION 1..9: note = Synthetic: M10
KTKCFQWQR

SEQ ID NO: 83 - protein, 8 aa, synthetic construct
  REGION 1..8: note = Synthetic: M10
KTKCFQWQ

SEQ ID NO: 84 - protein, 14 aa, synthetic construct
  REGION 1..14: note = Synthetic: M10
TKCFQWQRNMRKVR

SEQ ID NO: 85 - protein, 13 aa, synthetic construct
  REGION 1..13: note = Synthetic: M10
TKCFQWQRNMRKV

SEQ ID NO: 86 - protein, 12 aa, synthetic construct
  REGION 1..12: note = Synthetic: M10
TKCFQWQRNMRK

SEQ ID NO: 87 - protein, 11 aa, synthetic construct
  REGION 1..11: note = Synthetic: M10
TKCFQWQRNMR

SEQ ID NO: 88 - protein, 10 aa, synthetic construct
  REGION 1..10: note = Synthetic: M10
TKCFQWQRNM

SEQ ID NO: 89 - protein, 9 aa, synthetic construct
  REGION 1..9: note = Synthetic: M10
TKCFQWQRN

SEQ ID NO: 90 - protein, 8 aa, synthetic construct
  REGION 1..8: note = Synthetic: M10
TKCFQWQR

SEQ ID NO: 91 - protein, 13 aa, synthetic construct
  REGION 1..13: note = Synthetic: M10
KCFQWQRNMRKVR

SEQ ID NO: 92 - protein, 12 aa, synthetic construct
  REGION 1..12: note = Synthetic: M10
KCFQWQRNMRKV

SEQ ID NO: 93 - protein, 11 aa, synthetic construct
  REGION 1..11: note = Synthetic: M10
KCFQWQRNMRK

SEQ ID NO: 94 - protein, 10 aa, synthetic construct
  REGION 1..10: note = Synthetic: M10
KCFQWQRNMR

SEQ ID NO: 95 - protein, 9 aa, synthetic construct
  REGION 1..9: note = Synthetic: M10
KCFQWQRNM

SEQ ID NO: 96 - protein, 8 aa, synthetic construct
  REGION 1..8: note = Synthetic: M10
KCFQWQRN

SEQ ID NO: 97 - protein, 12 aa, synthetic construct
  REGION 1..12: note = Synthetic: M10
CFQWQRNMRKVR

SEQ ID NO: 98 - protein, 11 aa, synthetic construct
  REGION 1..11: note = Synthetic: M10
CFQWQRNMRKV

SEQ ID NO: 99 - protein, 10 aa, synthetic construct
  REGION 1..10: note = Synthetic: M10
CFQWQRNMRK

SEQ ID NO: 100 - protein, 9 aa, synthetic construct
  REGION 1..9: note = Synthetic: M10
CFQWQRNMR

SEQ ID NO: 101 - protein, 8 aa, synthetic construct
  REGION 1..8: note = Synthetic: M10
CFQWQRNM

SEQ ID NO: 102 - protein, 11 aa, synthetic construct
  REGION 1..11: note = Synthetic: M10
FQWQRNMRKVR

SEQ ID NO: 103 - protein, 10 aa, synthetic construct
  REGION 1..10: note = Synthetic: M10
FQWQRNMRKV

SEQ ID NO: 104 - protein, 9 aa, synthetic construct
  REGION 1..9: note = Synthetic: M10
FQWQRNMRK

SEQ ID NO: 105 - protein, 8 aa, synthetic construct
  REGION 1..8: note = Synthetic: M10
FQWQRNMR

SEQ ID NO: 106 - protein, 15 aa, synthetic construct
  REGION 1..15: note = Synthetic: M11
KTKCKQRQRNMRKVR

SEQ ID NO: 107 - protein, 14 aa, synthetic construct
  REGION 1..14: note = Synthetic: M11
KTKCKQRQRNMRKV

SEQ ID NO: 108 - protein, 13 aa, synthetic construct
  REGION 1..13: note = Synthetic: M11
KTKCKQRQRNMRK

SEQ ID NO: 109 - protein, 12 aa, synthetic construct
  REGION 1..12: note = Synthetic: M11
KTKCKQRQRNMR

SEQ ID NO: 110 - protein, 11 aa, synthetic construct
  REGION 1..11: note = Synthetic: M11
KTKCKQRQRNM

SEQ ID NO: 111 - protein, 10 aa, synthetic construct
  REGION 1..10: note = Synthetic: M11
KTKCKQRQRN

SEQ ID NO: 112 - protein, 9 aa, synthetic construct
  REGION 1..9: note = Synthetic: M11
KTKCKQRQR

SEQ ID NO: 113 - protein, 8 aa, synthetic construct
  REGION 1..8: note = Synthetic: M11
KTKCKQRQ

SEQ ID NO: 114 - protein, 14 aa, synthetic construct
  REGION 1..14: note = Synthetic: M11
TKCKQRQRNMRKVR

SEQ ID NO: 115 - protein, 13 aa, synthetic construct
  REGION 1..13: note = Synthetic: M11
TKCKQRQRNMRKV

SEQ ID NO: 116 - protein, 12 aa, synthetic construct
  REGION 1..12: note = Synthetic: M11
TKCKQRQRNMRK

SEQ ID NO: 117 - protein, 11 aa, synthetic construct
  REGION 1..11: note = Synthetic: M11
TKCKQRQRNMR

SEQ ID NO: 118 - protein, 10 aa, synthetic construct
  REGION 1..10: note = Synthetic: M11
TKCKQRQRNM

SEQ ID NO: 119 - protein, 9 aa, synthetic construct
  REGION 1..9: note = Synthetic: M11
TKCKQRQRN

SEQ ID NO: 120 - protein, 8 aa, synthetic construct
  REGION 1..8: note = Synthetic: M11
TKCKQRQR

SEQ ID NO: 121 - protein, 13 aa, synthetic construct
  REGION 1..13: note = Synthetic: M11
KCKQRQRNMRKVR

SEQ ID NO: 122 - protein, 12 aa, synthetic construct
  REGION 1..12: note = Synthetic: M11
KCKQRQRNMRKV

SEQ ID NO: 123 - protein, 11 aa, synthetic construct
  REGION 1..11: note = Synthetic: M11
KCKQRQRNMRK

SEQ ID NO: 124 - protein, 10 aa, synthetic construct
  REGION 1..10: note = Synthetic: M11
KCKQRQRNMR

SEQ ID NO: 125 - protein, 9 aa, synthetic construct
  REGION 1..9: note = Synthetic: M11
KCKQRQRNM

SEQ ID NO: 126 - protein, 8 aa, synthetic construct
  REGION 1..8: note = Synthetic: M11
KCKQRQRN

SEQ ID NO: 127 - protein, 12 aa, synthetic construct
  REGION 1..12: note = Synthetic: M11
CKQRQRNMRKVR

SEQ ID NO: 128 - protein, 11 aa, synthetic construct
  REGION 1..11: note = Synthetic: M11
CKQRQRNMRKV

SEQ ID NO: 129 - protein, 10 aa, synthetic construct
  REGION 1..10: note = Synthetic: M11
CKQRQRNMRK

SEQ ID NO: 130 - protein, 9 aa, synthetic construct
  REGION 1..9: note = Synthetic: M11
CKQRQRNMR

SEQ ID NO: 131 - protein, 8 aa, synthetic construct
  REGION 1..8: note = Synthetic: M11
CKQRQRNM

SEQ ID NO: 132 - protein, 11 aa, synthetic construct
  REGION 1..11: note = Synthetic: M11
KQRQRNMRKVR

SEQ ID NO: 133 - protein, 10 aa, synthetic construct
  REGION 1..10: note = Synthetic: M11
KQRQRNMRKV

SEQ ID NO: 134 - protein, 9 aa, synthetic construct
  REGION 1..9: note = Synthetic: M11
KQRQRNMRK

SEQ ID NO: 135 - protein, 8 aa, synthetic construct
  REGION 1..8: note = Synthetic: M11
KQRQRNMR

SEQ ID NO: 136 - protein, 10 aa, synthetic construct
  REGION 1..10: note = Synthetic: M11
QRQRNMRKVR

SEQ ID NO: 137 - protein, 9 aa, synthetic construct
  REGION 1..9: note = Synthetic: M11
QRQRNMRKV

SEQ ID NO: 138 - protein, 8 aa, synthetic construct
  REGION 1..8: note = Synthetic: M11
QRQRNMRK

SEQ ID NO: 139 - protein, 9 aa, synthetic construct
  REGION 1..9: note = Synthetic: M11
RQRNMRKVR

SEQ ID NO: 140 - protein, 8 aa, synthetic construct
  REGION 1..8: note = Synthetic: M11
RQRNMRKV

SEQ ID NO: 141 - protein, 11 aa, synthetic construct
  REGION 1..11: note = Synthetic: M12
KQRQRNMRKVR

SEQ ID NO: 142 - protein, 15 aa, synthetic construct
  REGION 1..15: note = Synthetic: M13
MRKVRGPPKSCRKRD

SEQ ID NO: 143 - protein, 14 aa, synthetic construct
  REGION 1..14: note = Synthetic: M13
MRKVRGPPKSCRKR

SEQ ID NO: 144 - protein, 13 aa, synthetic construct
  REGION 1..13: note = Synthetic: M13
MRKVRGPPKSCRK

SEQ ID NO: 145 - protein, 12 aa, synthetic construct
  REGION 1..12: note = Synthetic: M13
MRKVRGPPKSCR

SEQ ID NO: 146 - protein, 11 aa, synthetic construct
  REGION 1..11: note = Synthetic: M13
MRKVRGPPKSC

SEQ ID NO: 147 - protein, 10 aa, synthetic construct
  REGION 1..10: note = Synthetic: M13
MRKVRGPPKS

SEQ ID NO: 148 - protein, 9 aa, synthetic construct
  REGION 1..9: note = Synthetic: M13
MRKVRGPPK

SEQ ID NO: 149 - protein, 8 aa, synthetic construct
  REGION 1..8: note = Synthetic: M13
MRKVRGPP

SEQ ID NO: 150 - protein, 14 aa, synthetic construct
  REGION 1..14: note = Synthetic: M13
RKVRGPPKSCRKRD

SEQ ID NO: 151 - protein, 13 aa, synthetic construct
  REGION 1..13: note = Synthetic: M13
RKVRGPPKSCRKR

SEQ ID NO: 152 - protein, 12 aa, synthetic construct
  REGION 1..12: note = Synthetic: M13
RKVRGPPKSCRK

SEQ ID NO: 153 - protein, 11 aa, synthetic construct
  REGION 1..11: note = Synthetic: M13
RKVRGPPKSCR

SEQ ID NO: 154 - protein, 10 aa, synthetic construct
  REGION 1..10: note = Synthetic: M13
RKVRGPPKSC

SEQ ID NO: 155 - protein, 9 aa, synthetic construct
  REGION 1..9: note = Synthetic: M13
RKVRGPPKS

SEQ ID NO: 156 - protein, 8 aa, synthetic construct
  REGION 1..8: note = Synthetic: M13
RKVRGPPK

SEQ ID NO: 157 - protein, 13 aa, synthetic construct
  REGION 1..13: note = Synthetic: M13
KVRGPPKSCRKRD

SEQ ID NO: 158 - protein, 12 aa, synthetic construct
  REGION 1..12: note = Synthetic: M13
KVRGPPKSCRKR

SEQ ID NO: 159 - protein, 11 aa, synthetic construct
  REGION 1..11: note = Synthetic: M13
KVRGPPKSCRK

SEQ ID NO: 160 - protein, 10 aa, synthetic construct
  REGION 1..10: note = Synthetic: M13
KVRGPPKSCR

SEQ ID NO: 161 - protein, 9 aa, synthetic construct
  REGION 1..9: note = Synthetic: M13
KVRGPPKSC

SEQ ID NO: 162 - protein, 8 aa, synthetic construct
  REGION 1..8: note = Synthetic: M13
KVRGPPKS

SEQ ID NO: 163 - protein, 12 aa, synthetic construct
  REGION 1..12: note = Synthetic: M13
VRGPPKSCRKRD

SEQ ID NO: 164 - protein, 11 aa, synthetic construct
  REGION 1..11: note = Synthetic: M13
VRGPPKSCRKR

SEQ ID NO: 165 - protein, 10 aa, synthetic construct
  REGION 1..10: note = Synthetic: M13
VRGPPKSCRK

SEQ ID NO: 166 - protein, 9 aa, synthetic construct
  REGION 1..9: note = Synthetic: M13
VRGPPKSCR

SEQ ID NO: 167 - protein, 8 aa, synthetic construct
  REGION 1..8: note = Synthetic: M13
VRGPPKSC

SEQ ID NO: 168 - protein, 11 aa, synthetic construct
  REGION 1..11: note = Synthetic: M13
RGPPKSCRKRD

SEQ ID NO: 169 - protein, 10 aa, synthetic construct
  REGION 1..10: note = Synthetic: M13
RGPPKSCRKR

SEQ ID NO: 170 - protein, 9 aa, synthetic construct
  REGION 1..9: note = Synthetic: M13
RGPPKSCRK

SEQ ID NO: 171 - protein, 8 aa, synthetic construct
  REGION 1..8: note = Synthetic: M13
RGPPKSCR

SEQ ID NO: 172 - protein, 10 aa, synthetic construct
  REGION 1..10: note = Synthetic: M13
GPPKSCRKRD

SEQ ID NO: 173 - protein, 9 aa, synthetic construct
  REGION 1..9: note = Synthetic: M13
GPPKSCRKR

SEQ ID NO: 174 - protein, 8 aa, synthetic construct
  REGION 1..8: note = Synthetic: M13
GPPKSCRK

SEQ ID NO: 175 - protein, 9 aa, synthetic construct
  REGION 1..9: note = Synthetic: M13
PPKSCRKRD

SEQ ID NO: 176 - protein, 8 aa, synthetic construct
  REGION 1..8: note = Synthetic: M13
PPKSCRKR

SEQ ID NO: 177 - protein, 11 aa, synthetic construct
  REGION 1..11: note = Synthetic: M14
RGPPKSCRKRD

SEQ ID NO: 178 - protein, 15 aa, synthetic construct
  REGION 1..15: note = Synthetic: M15
RQPRTHKYAVRVVKK

SEQ ID NO: 179 - protein, 14 aa, synthetic construct
  REGION 1..14: note = Synthetic: M15
RQPRTHKYAVRVVK

SEQ ID NO: 180 - protein, 13 aa, synthetic construct
  REGION 1..13: note = Synthetic: M15
RQPRTHKYAVRVV

SEQ ID NO: 181 - protein, 12 aa, synthetic construct
  REGION 1..12: note = Synthetic: M15
RQPRTHKYAVRV

SEQ ID NO: 182 - protein, 11 aa, synthetic construct
  REGION 1..11: note = Synthetic: M15
RQPRTHKYAVR

SEQ ID NO: 183 - protein, 10 aa, synthetic construct
  REGION 1..10: note = Synthetic: M15
RQPRTHKYAV

SEQ ID NO: 184 - protein, 9 aa, synthetic construct
  REGION 1..9: note = Synthetic: M15
RQPRTHKYA

SEQ ID NO: 185 - protein, 8 aa, synthetic construct
  REGION 1..8: note = Synthetic: M15
RQPRTHKY

SEQ ID NO: 186 - protein, 14 aa, synthetic construct
  REGION 1..14: note = Synthetic: M15
QPRTHKYAVRVVKK

SEQ ID NO: 187 - protein, 13 aa, synthetic construct
  REGION 1..13: note = Synthetic: M15
QPRTHKYAVRVVK

SEQ ID NO: 188 - protein, 12 aa, synthetic construct
  REGION 1..12: note = Synthetic: M15
QPRTHKYAVRVV

SEQ ID NO: 189 - protein, 11 aa, synthetic construct
  REGION 1..11: note = Synthetic: M15
QPRTHKYAVRV

SEQ ID NO: 190 - protein, 10 aa, synthetic construct
  REGION 1..10: note = Synthetic: M15
QPRTHKYAVR

SEQ ID NO: 191 - protein, 9 aa, synthetic construct
  REGION 1..9: note = Synthetic: M15
QPRTHKYAV

SEQ ID NO: 192 - protein, 8 aa, synthetic construct
  REGION 1..8: note = Synthetic: M15
QPRTHKYA

SEQ ID NO: 193 - protein, 13 aa, synthetic construct
  REGION 1..13: note = Synthetic: M15
PRTHKYAVRVVKK

SEQ ID NO: 194 - protein, 12 aa, synthetic construct
  REGION 1..12: note = Synthetic: M15
PRTHKYAVRVVK

SEQ ID NO: 195 - protein, 11 aa, synthetic construct
  REGION 1..11: note = Synthetic: M15
PRTHKYAVRVV

SEQ ID NO: 196 - protein, 10 aa, synthetic construct
  REGION 1..10: note = Synthetic: M15
PRTHKYAVRV

SEQ ID NO: 197 - protein, 9 aa, synthetic construct
  REGION 1..9: note = Synthetic: M15
PRTHKYAVR

SEQ ID NO: 198 - protein, 8 aa, synthetic construct
  REGION 1..8: note = Synthetic: M15
PRTHKYAV

SEQ ID NO: 199 - protein, 12 aa, synthetic construct
  REGION 1..12: note = Synthetic: M15
RTHKYAVRVVKK

SEQ ID NO: 200 - protein, 11 aa, synthetic construct
  REGION 1..11: note = Synthetic: M15
RTHKYAVRVVK

SEQ ID NO: 201 - protein, 10 aa, synthetic construct
  REGION 1..10: note = Synthetic: M15
RTHKYAVRVV

SEQ ID NO: 202 - protein, 9 aa, synthetic construct
  REGION 1..9: note = Synthetic: M15
RTHKYAVRV

SEQ ID NO: 203 - protein, 8 aa, synthetic construct
  REGION 1..8: note = Synthetic: M15
RTHKYAVR

SEQ ID NO: 204 - protein, 11 aa, synthetic construct
  REGION 1..11: note = Synthetic: M15
THKYAVRVVKK

SEQ ID NO: 205 - protein, 10 aa, synthetic construct
  REGION 1..10: note = Synthetic: M15
THKYAVRVVK

SEQ ID NO: 206 - protein, 9 aa, synthetic construct
  REGION 1..9: note = Synthetic: M15
THKYAVRVV

SEQ ID NO: 207 - protein, 8 aa, synthetic construct
  REGION 1..8: note = Synthetic: M15
THKYAVRV

SEQ ID NO: 208 - protein, 10 aa, synthetic construct
  REGION 1..10: note = Synthetic: M15
HKYAVRVVKK

SEQ ID NO: 209 - protein, 9 aa, synthetic construct
  REGION 1..9: note = Synthetic: M15
HKYAVRVVK

SEQ ID NO: 210 - protein, 8 aa, synthetic construct
  REGION 1..8: note = Synthetic: M15
HKYAVRVV

SEQ ID NO: 211 - protein, 9 aa, synthetic construct
  REGION 1..9: note = Synthetic: M15
KYAVRVVKK

SEQ ID NO: 212 - protein, 8 aa, synthetic construct
  REGION 1..8: note = Synthetic: M15
KYAVRVVK

SEQ ID NO: 213 - protein, 10 aa, synthetic construct
  REGION 1..10: note = Synthetic: M16
HKYAVRVVKK

SEQ ID NO: 214 - protein, 9 aa, synthetic construct
  REGION 1..9: note = Synthetic: M16
HKYAVRVVK

SEQ ID NO: 215 - protein, 8 aa, synthetic construct
  REGION 1..8: note = Synthetic: M16
HKYAVRVV

SEQ ID NO: 216 - protein, 9 aa, synthetic construct
  REGION 1..9: note = Synthetic: M16
KYAVRVVKK

SEQ ID NO: 217 - protein, 8 aa, synthetic construct
  REGION 1..8: note = Synthetic: M16
KYAVRVVK

SEQ ID NO: 218 - protein, 15 aa, synthetic construct
  REGION 1..15: note = Synthetic: M17
ATKCFQWQRNMRKVR

SEQ ID NO: 219 - protein, 11 aa, synthetic construct
  REGION 1..11: note = Synthetic: M18
FQWQRNMRKVR

SEQ ID NO: 220 - protein, 15 aa, synthetic construct
  REGION 1..15: note = Synthetic: M19
MRKVRGPPVSCIKRD

SEQ ID NO: 221 - protein, 14 aa, synthetic construct
  REGION 1..14: note = Synthetic: M19
MRKVRGPPVSCIKR

SEQ ID NO: 222 - protein, 13 aa, synthetic construct
  REGION 1..13: note = Synthetic: M19
MRKVRGPPVSCIK

SEQ ID NO: 223 - protein, 12 aa, synthetic construct
  REGION 1..12: note = Synthetic: M19
MRKVRGPPVSCI

SEQ ID NO: 224 - protein, 11 aa, synthetic construct
  REGION 1..11: note = Synthetic: M19
MRKVRGPPVSC

SEQ ID NO: 225 - protein, 10 aa, synthetic construct
  REGION 1..10: note = Synthetic: M19
MRKVRGPPVS

SEQ ID NO: 226 - protein, 9 aa, synthetic construct
  REGION 1..9: note = Synthetic: M19
MRKVRGPPV

SEQ ID NO: 227 - protein, 8 aa, synthetic construct
  REGION 1..8: note = Synthetic: M19
MRKVRGPP

SEQ ID NO: 228 - protein, 14 aa, synthetic construct
  REGION 1..14: note = Synthetic: M19
RKVRGPPVSCIKRD

SEQ ID NO: 229 - protein, 13 aa, synthetic construct
  REGION 1..13: note = Synthetic: M19
RKVRGPPVSCIKR

SEQ ID NO: 230 - protein, 12 aa, synthetic construct
  REGION 1..12: note = Synthetic: M19
RKVRGPPVSCIK

SEQ ID NO: 231 - protein, 11 aa, synthetic construct
  REGION 1..11: note = Synthetic: M19
RKVRGPPVSCI

SEQ ID NO: 232 - protein, 10 aa, synthetic construct
  REGION 1..10: note = Synthetic: M19
RKVRGPPVSC

SEQ ID NO: 233 - protein, 9 aa, synthetic construct
  REGION 1..9: note = Synthetic: M19
RKVRGPPVS

SEQ ID NO: 234 - protein, 8 aa, synthetic construct
  REGION 1..8: note = Synthetic: M19
RKVRGPPV

SEQ ID NO: 235 - protein, 13 aa, synthetic construct
  REGION 1..13: note = Synthetic: M19
KVRGPPVSCIKRD

SEQ ID NO: 236 - protein, 12 aa, synthetic construct
  REGION 1..12: note = Synthetic: M19
KVRGPPVSCIKR

SEQ ID NO: 237 - protein, 11 aa, synthetic construct
  REGION 1..11: note = Synthetic: M19
KVRGPPVSCIK

SEQ ID NO: 238 - protein, 10 aa, synthetic construct
  REGION 1..10: note = Synthetic: M19
KVRGPPVSCI

SEQ ID NO: 239 - protein, 9 aa, synthetic construct
  REGION 1..9: note = Synthetic: M19
KVRGPPVSC

SEQ ID NO: 240 - protein, 8 aa, synthetic construct
  REGION 1..8: note = Synthetic: M19
KVRGPPVS

SEQ ID NO: 241 - protein, 12 aa, synthetic construct
  REGION 1..12: note = Synthetic: M19
VRGPPVSCIKRD

SEQ ID NO: 242 - protein, 11 aa, synthetic construct
  REGION 1..11: note = Synthetic: M19
VRGPPVSCIKR

SEQ ID NO: 243 - protein, 10 aa, synthetic construct
  REGION 1..10: note = Synthetic: M19
VRGPPVSCIK

SEQ ID NO: 244 - protein, 9 aa, synthetic construct
  REGION 1..9: note = Synthetic: M19
VRGPPVSCI

SEQ ID NO: 245 - protein, 8 aa, synthetic construct
  REGION 1..8: note = Synthetic: M19
VRGPPVSC

SEQ ID NO: 246 - protein, 11 aa, synthetic construct
  REGION 1..11: note = Synthetic: M19
RGPPVSCIKRD

SEQ ID NO: 247 - protein, 10 aa, synthetic construct
  REGION 1..10: note = Synthetic: M19
RGPPVSCIKR

SEQ ID NO: 248 - protein, 9 aa, synthetic construct
  REGION 1..9: note = Synthetic: M19
RGPPVSCIK

SEQ ID NO: 249 - protein, 8 aa, synthetic construct
  REGION 1..8: note = Synthetic: M19
RGPPVSCI

SEQ ID NO: 250 - protein, 10 aa, synthetic construct
  REGION 1..10: note = Synthetic: M19
GPPVSCIKRD

SEQ ID NO: 251 - protein, 9 aa, synthetic construct
  REGION 1..9: note = Synthetic: M19
GPPVSCIKR

SEQ ID NO: 252 - protein, 8 aa, synthetic construct
  REGION 1..8: note = Synthetic: M19
GPPVSCIK

SEQ ID NO: 253 - protein, 9 aa, synthetic construct
  REGION 1..9: note = Synthetic: M19
PPVSCIKRD

SEQ ID NO: 254 - protein, 8 aa, synthetic construct
  REGION 1..8: note = Synthetic: M19
PPVSCIKR

SEQ ID NO: 255 - protein, 11 aa, synthetic construct
  REGION 1..11: note = Synthetic: M20
RGPPVSCIKRD

SEQ ID NO: 256 - protein, 15 aa, synthetic construct
  REGION 1..15: note = Synthetic: M21
RQPRTHYYAVAVVKK

SEQ ID NO: 257 - protein, 14 aa, synthetic construct
  REGION 1..14: note = Synthetic: M21
RQPRTHYYAVAVVK

SEQ ID NO: 258 - protein, 13 aa, synthetic construct
  REGION 1..13: note = Synthetic: M21
RQPRTHYYAVAVV

SEQ ID NO: 259 - protein, 12 aa, synthetic construct
  REGION 1..12: note = Synthetic: M21
RQPRTHYYAVAV

SEQ ID NO: 260 - protein, 11 aa, synthetic construct
  REGION 1..11: note = Synthetic: M21
RQPRTHYYAVA

SEQ ID NO: 261 - protein, 10 aa, synthetic construct
  REGION 1..10: note = Synthetic: M21
RQPRTHYYAV

SEQ ID NO: 262 - protein, 9 aa, synthetic construct
  REGION 1..9: note = Synthetic: M21
RQPRTHYYA

SEQ ID NO: 263 - protein, 8 aa, synthetic construct
  REGION 1..8: note = Synthetic: M21
RQPRTHYY

SEQ ID NO: 264 - protein, 14 aa, synthetic construct
  REGION 1..14: note = Synthetic: M21
QPRTHYYAVAVVKK

SEQ ID NO: 265 - protein, 13 aa, synthetic construct
  REGION 1..13: note = Synthetic: M21
QPRTHYYAVAVVK

SEQ ID NO: 266 - protein, 12 aa, synthetic construct
  REGION 1..12: note = Synthetic: M21
QPRTHYYAVAVV

SEQ ID NO: 267 - protein, 11 aa, synthetic construct
  REGION 1..11: note = Synthetic: M21
QPRTHYYAVAV

SEQ ID NO: 268 - protein, 10 aa, synthetic construct
  REGION 1..10: note = Synthetic: M21
QPRTHYYAVA

SEQ ID NO: 269 - protein, 9 aa, synthetic construct
  REGION 1..9: note = Synthetic: M21
QPRTHYYAV

SEQ ID NO: 270 - protein, 8 aa, synthetic construct
  REGION 1..8: note = Synthetic: M21
QPRTHYYA

SEQ ID NO: 271 - protein, 13 aa, synthetic construct
  REGION 1..13: note = Synthetic: M21
PRTHYYAVAVVKK

SEQ ID NO: 272 - protein, 12 aa, synthetic construct
  REGION 1..12: note = Synthetic: M21
PRTHYYAVAVVK

SEQ ID NO: 273 - protein, 11 aa, synthetic construct
  REGION 1..11: note = Synthetic: M21
PRTHYYAVAVV

SEQ ID NO: 274 - protein, 10 aa, synthetic construct
  REGION 1..10: note = Synthetic: M21
PRTHYYAVAV

SEQ ID NO: 275 - protein, 9 aa, synthetic construct
  REGION 1..9: note = Synthetic: M21
PRTHYYAVA

SEQ ID NO: 276 - protein, 8 aa, synthetic construct
  REGION 1..8: note = Synthetic: M21
PRTHYYAV

SEQ ID NO: 277 - protein, 12 aa, synthetic construct
  REGION 1..12: note = Synthetic: M21
RTHYYAVAVVKK

SEQ ID NO: 278 - protein, 11 aa, synthetic construct
  REGION 1..11: note = Synthetic: M21
RTHYYAVAVVK

SEQ ID NO: 279 - protein, 10 aa, synthetic construct
  REGION 1..10: note = Synthetic: M21
RTHYYAVAVV

SEQ ID NO: 280 - protein, 9 aa, synthetic construct
  REGION 1..9: note = Synthetic: M21
RTHYYAVAV

SEQ ID NO: 281 - protein, 8 aa, synthetic construct
  REGION 1..8: note = Synthetic: M21
RTHYYAVA

SEQ ID NO: 282 - protein, 11 aa, synthetic construct
  REGION 1..11: note = Synthetic: M21
THYYAVAVVKK

SEQ ID NO: 283 - protein, 10 aa, synthetic construct
  REGION 1..10: note = Synthetic: M21
THYYAVAVVK

SEQ ID NO: 284 - protein, 9 aa, synthetic construct
  REGION 1..9: note = Synthetic: M21
THYYAVAVV

SEQ ID NO: 285 - protein, 8 aa, synthetic construct
  REGION 1..8: note = Synthetic: M21
THYYAVAV

SEQ ID NO: 286 - protein, 10 aa, synthetic construct
  REGION 1..10: note = Synthetic: M21
HYYAVAVVKK

SEQ ID NO: 287 - protein, 9 aa, synthetic construct
  REGION 1..9: note = Synthetic: M21
HYYAVAVVK

SEQ ID NO: 288 - protein, 8 aa, synthetic construct
  REGION 1..8: note = Synthetic: M21
HYYAVAVV

SEQ ID NO: 289 - protein, 9 aa, synthetic construct
  REGION 1..9: note = Synthetic: M21
YYAVAVVKK

SEQ ID NO: 290 - protein, 8 aa, synthetic construct
  REGION 1..8: note = Synthetic: M21
YYAVAVVK

SEQ ID NO: 291 - protein, 9 aa, synthetic construct
  REGION 1..9: note = Synthetic: M22
HYYAVAVVK

SEQ ID NO: 292 - protein, 8 aa, synthetic construct
  REGION 1..8: note = Synthetic: M22
HYYAVAVV

SEQ ID NO: 293 - protein, 7 aa, synthetic construct
  REGION 1..7: note = Synthetic: M22
HYYAVAV

SEQ ID NO: 294 - protein, 9 aa, synthetic construct
  REGION 1..9: note = Synthetic: M22
YYAVAVVKK

SEQ ID NO: 295 - protein, 8 aa, synthetic construct
  REGION 1..8: note = Synthetic: M22
YYAVAVVK

SEQ ID NO: 296 - protein, 182 aa, Camelus dromedarius
  REGION 1..182: note = misc_feature - P79139 - camel k-casein protein
MKSFFLVVTILALTLPFLGAEVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 297 - protein, 710 aa, Homo sapiens
  REGION 1..710: note = misc_feature - P02788 - human Lactotransferrin protein
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 298 - protein, 223 aa, Capra hircus
  REGION 1..223: note = misc_feature - P33049 - goat a-S2-casein protein
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 299 - protein, 1927 aa, Homo sapiens
  REGION 1..1927: note = misc_feature - P09848 - human Lactase-phlorizin hydrolase
MELSWHVVFIALLSFSCWGSDWESDRNFISTAGPLTNDLLHNLSGLLGDQSSNFVAGDKDMYVCHQPLPTFLPEYFSSLHASQITHYKVFLSWAQLLPAGSTQNPDEKTVQCYRRLLKALKTARLQPMVILHHQTLPASTLRRTEAFADLFADYATFAFHSFGDLVGIWFTFSDLEEVIKELPHQESRASQLQTLSDAHRKAYEIYHESYAFQGGKLSVVLRAEDIPELLLEPPISALAQDTVDFLSLDLSYECQNEASLRQKLSKLQTIEPKVKVFIFNLKLPDCPSTMKNPASLLFSLFEAINKDQVLTIGFDINEFLSCSSSSKKSMSCSLTGSLALQPDQQQDHETTDSSPASAYQRIWEAFANQSRAERDAFLQDTFPEGFLWGASTGAFNVEGGWAEGGRGVSIWDPRRPLNTTEGQATLEVASDSYHKVASDVALLCGLRAQVYKFSISWSRIFPMGHGSSPSLPGVAYYNKLIDRLQDAGIEPMATLFHWDLPQALQDHGGWQNESVVDAFLDYAAFCFSTFGDRVKLWVTFHEPWVMSYAGYGTGQHPPGISDPGVASFKVAHLVLKAHARTWHHYNSHHRPQQQGHVGIVLNSDWAEPLSPERPEDLRASERFLHFMLGWFAHPVFVDGDYPATLRTQIQQMNRQCSHPVAQLPEFTEAEKQLLKGSADFLGLSHYTSRLISNAPQNTCIPSYDTIGGFSQHVNHVWPQTSSSWIRVVPWGIRRLLQFVSLEYTRGKVPIYLAGNGMPIGESENLFDDSLRVDYFNQYINEVLKAIKEDSVDVRSYIARSLIDGFEGPSGYSQRFGLHHVNFSDSSKSRTPRKSAYFFTSIIEKNGFLTKGAKRLLPPNTVNLPSKVRAFTFPSEVPSKAKVVWEKFSSQPKFERDLFYHGTFRDDFLWGVSSSAYQIEGAWDADGKGPSIWDNFTHTPGSNVKDNATGDIACDSYHQLDADLNMLRALKVKAYRFSISWSRIFPTGRNSSINSHGVDYYNRLINGLVASNIFPMVTLFHWDLPQALQDIGGWENPALIDLFDSYADFCFQTFGDRVKFWMTFNEPMYLAWLGYGSGEFPPGVKDPGWAPYRIAHAVIKAHARVYHTYDEKYRQEQKGVISLSLSTHWAEPKSPGVPRDVEAADRMLQFSLGWFAHPIFRNGDYPDTMKWKVGNRSELQHLATSRLPSFTEEEKRFIRATADVFCLNTYYSRIVQHKTPRLNPPSYEDDQEMAEEEDPSWPSTAMNRAAPWGTRRLLNWIKEEYGDIPIYITENGVGLTNPNTEDTDRIFYHKTYINEALKAYRLDGIDLRGYVAWSLMDNFEWLNGYTVKFGLYHVDFNNTNRPRTARASARYYTEVITNNGMPLAREDEFLYGRFPEGFIWSAASAAYQIEGAWRADGKGLSIWDTFSHTPLRVENDAIGDVACDSYHKIAEDLVTLQNLGVSHYRFSISWSRILPDGTTRYINEAGLNYYVRLIDTLLAASIQPQVTIYHWDLPQTLQDVGGWENETIVQRFKEYADVLFQRLGDKVKFWITLNEPFVIAYQGYGYGTAAPGVSNRPGTAPYIVGHNLIKAHAEAWHLYNDVYRASQGGVISITISSDWAEPRDPSNQEDVEAARRYVQFMGGWFAHPIFKNGDYNEVMKTRIRDRSLAAGLNKSRLPEFTESEKRRINGTYDFFGFNHYTTVLAYNLNYATAISSFDADRGVASIADRSWPDSGSFWLKMTPFGFRRILNWLKEEYNDPPIYVTENGVSQREETDLNDTARIYYLRTYINEALKAVQDKVDLRGYTVWSAMDNFEWATGFSERFGLHFVNYSDPSLPRIPKASAKFYASVVRCNGFPDPATGPHACLHQPDAGPTISPVRQEEVQFLGLMLGTTEAQTALYVLFSLVLLGVCGLAFLSYKYCKRSKQGKTQRSQQELSPVSSF

SEQ ID NO: 300 - protein, 222 aa, Capra hircus
  REGION 1..222: note = misc_feature - P33048 - goat B-casein protein
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 301 - protein, 148 aa, Bos taurus
  REGION 1..148: note = misc_feature - Q6B411 - bovine Lysozyme C isozyme protein
MKALLIVGLLLLSVAVQGKKFQRCELARTLKKLGLDGYRGVSLANWVCLARWESNYNTRATNYNRGDKSTDYGIFQINSRWWCNDGKTPKAVNACRIPCSALLKDDITQAVACAKRVVRDPQGIKAWVAWRNKCQNRDLRSYVQGCRV